(19) United States
(12) Patent Application Publication
(10) Pub. No.: US 2025/0325672 A1
(43) Pub. Date: Oct. 23, 2025

(54) ANTI-INFECTIVE BICYCLIC PEPTIDE LIGANDS

(71) Applicants: Bicycle Tx Limited, Cambridge (GB); United Kingdom Research and Innovation, Swindon, Wiltshire (GB)

(72) Inventors: Nicholas KEEN, Carlisle, MA (US); Katerine VAN RIETSCHOTEN, Cambridge (GB); Katie GAYNOR, Cambridge (GB); Liuhong CHEN, Cambridge (GB); Naximilian HARMAN, Cambridge (GB); Michael SKYNNER, Cambridge (GB); Paul BESWICK, Cambridge (GB); Mehdi AMOURA, Cambridge (GB); Simone GIORGI, Cambridge (GB); Giulia LATTANZI, Cambridge (GB); Iain LINGARD, Cambridge (GB)

(21) Appl. No.: 18/271,330

(22) PCT Filed: Jan. 10, 2022
(86) PCT No.: PCT/GB2022/050031

§ 371 Date: Jul. 7, 2023

Related U.S. Application Data
(60) Provisional application No. 63/132,361, filed on Dec. 30, 2020.
(60) Provisional application No. 63/164,069, filed on Mar. 22, 2021.

(51) Int. Cl. A61K 47/54; A61P 31/14

(52) U.S. Cl. CPC A61K 47/545; A61P 31/14

(57) ABSTRACT

The present invention relates to polypeptides which are covalently bound to molecular scaffolds such that two or more peptide loops are subtended between attachment points to the scaffold. In particular, the invention describes peptides which are high affinity binders of severe acute respiratory syndrome coronavirus 2 (SARS-COV-2), particularly the spike protein S1 of SARS-COV-2. The invention also includes pharmaceutical compositions comprising said polypeptides and to the use of said polypeptides in suppressing or treating a disease or disorder mediated by infection of SARS-COV-2 or for providing prophylaxis to a subject at risk of infection of SARS-COV-2.

### CROSS REFERENCE TO RELATED APPLICATIONS

This application is the national stage of International Patent Application No. PCT/GB2022/050031, filed Jan. 10, 2022, which claims the benefit of U.S. Provisional Application No. 63/164,068, filed Mar. 22, 2021, and U.S. Provisional Application No. 63/135,361, filed Jan. 8, 2021, the entire contents of each of which are incorporated herein by reference.

### SEQUENCE LISTING

The instant application contains a Sequence Listing in ASCII format which has been submitted electronically and is hereby incorporated by reference in its entirety. Said copy, created on Mar. 21, 2024, is named 201248_SL.txt and is 54.5 kilobytes in size.

### FIELD OF THE INVENTION

The present invention relates to polypeptides which are covalently bound to molecular scaffolds such that two or more peptide loops are subtended between attachment points to the scaffold. In particular, the invention describes peptides which are high affinity binders of severe acute respiratory syndrome coronavirus 2 (SARS-COV-2), particularly the spike protein S1 of SARS-COV-2. The invention also includes pharmaceutical compositions comprising said polypeptides and to the use of said polypeptides in suppressing or treating a disease or disorder mediated by infection of SARS-COV-2 or for providing prophylaxis to a subject at risk of infection of SARS-COV-2.

### BACKGROUND OF THE INVENTION

Coronavirus disease 2019 (COVID-19) is an infectious disease caused by severe acute respiratory syndrome coronavirus 2 (SARS-COV-2). The disease was first identified in December 2019 in Wuhan, the capital of China's Hubei province, and spread globally, resulting in a pandemic. Common symptoms include fever, cough, and shortness of breath. Other symptoms may include fatigue, muscle pain, diarrhea, sore throat, loss of smell, and abdominal pain. The time from exposure to onset of symptoms is typically around five days but may range from two to fourteen days. While the majority of cases result in mild symptoms, some progress to viral pneumonia and multi-organ failure. As of 6 Jan. 2021, more than 86 million cases have been reported globally, resulting in more than 1.8 million deaths.

The virus is primarily spread between people during close contact, often via droplets produced by coughing, sneezing, or talking. While these droplets are produced when breathing out, they usually fall to the ground or onto surfaces rather than being infectious over long distances. People may also become infected by touching a contaminated surface and then their face. The virus can survive on surfaces for up to 72 hours. It is most contagious during the first three days after the onset of symptoms, although spread may be possible before symptoms appear and in later stages of the disease.

Currently, there is no vaccine or specific antiviral treatment for COVID-19. Management involves treatment of symptoms, supportive care, isolation, and experimental measures. The World Health Organization (WHO) declared the 2019-2020 coronavirus outbreak a Public Health Emergency of International Concern (PHEIC) on 30 Jan. 2020 and a pandemic on 11 Mar. 2020. Local transmission of the disease has been recorded in many countries across all six WHO regions.

There is therefore a great need to provide an effective prophylactic and/or therapeutic treatment intended to avoid or ameliorate the symptoms associated with infection of SARS-CoV-2, such as COVID-19.

### SUMMARY OF THE INVENTION

According to a first aspect of the invention, there is provided a peptide ligand specific for severe acute respiratory syndrome coronavirus 2 (SARS-COV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a further aspect of the invention, there is provided a pharmaceutical composition comprising the peptide ligand as defined herein in combination with one or more pharmaceutically acceptable excipients.

According to a further aspect of the invention, there is provided the peptide ligand as defined herein for use in suppressing or treating a disease or disorder mediated by infection of SARS-CoV-2 or for providing prophylaxis to a subject at risk of infection of SARS-COV-2.

### DETAILED DESCRIPTION OF THE INVENTION

According to a first aspect of the invention, there is provided a peptide ligand specific for severe acute respiratory syndrome coronavirus 2 (SARS-COV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

In one embodiment, said peptide ligand is specific for the spike protein of SARS-COV-2. The spike protein (S protein) is a large type I transmembrane protein of SARS-COV-2. This protein is highly glycosylated as it contains 21 to 35 N-glycosylation sites. Spike proteins assemble into trimers on the virion surface to form the distinctive “corona”, or crown-like appearance. The ectodomain of all CoV spike proteins share the same organization in two domains: a N-terminal domain named S1 that is responsible for receptor binding and a C-terminal S2 domain responsible for fusion. CoV diversity is reflected in the variable spike proteins (S proteins), which have evolved into forms differing in their receptor interactions and their response to various environmental triggers of virus-cell membrane fusion.

In a further embodiment, said peptide ligand binds to either the S1 of S2 domain of the spike protein (S protein). In a yet further embodiment, said peptide ligand binds to the S1 domain of the spike protein (S1 protein). Without being bound by theory it is believed that binding to the S1 domain of SARS-COV-2, namely the receptor binding domain of SARS-COV-2, will prevent the virus from binding to its target (thought to be ACE2 bound to the surface of lung airway cells) to enter tissue and cause disease.

In one embodiment, said loop sequences comprise 2, 3, 4, 5, 6, 7 or 8 amino acids.

In one embodiment, said loop sequences comprise three reactive groups separated by two loop sequences one of which consists of 3 amino acids and the other of which consists of 6 amino acids.

In a further embodiment, said loop sequences comprise three reactive groups separated by two loop sequences one of which consists of 3 amino acids and the other of which consists of 6 amino acids and the bicyclic peptide ligand comprises an amino acid sequence which is selected from:

```
  (SEQ ID NO: 1)
CᵢHHACᵢᵢPILTGWCᵢᵢᵢ;
  (SEQ ID NO: 6)
CᵢPHACᵢᵢPSLWGWCᵢᵢᵢ;
  (SEQ ID NO: 7)
CᵢLHACᵢᵢPRLTHWCᵢᵢᵢ;
  (SEQ ID NO: 8)
CᵢLHACᵢᵢQYLWGYCᵢᵢᵢ;
  (SEQ ID NO: 9)
CᵢSHACᵢᵢPRLFGWCᵢᵢᵢ;
  (SEQ ID NO: 10)
CᵢQHACᵢᵢPYLWDYCᵢᵢᵢ;
  (SEQ ID NO: 58)
CᵢPFACᵢᵢHKLYGWCᵢᵢᵢ;
  (SEQ ID NO: 59)
CᵢMKACᵢᵢPYLYGWCᵢᵢᵢ;
  (SEQ ID NO: 60)
CᵢRHACᵢᵢTHLYGHCᵢᵢᵢ;
  (SEQ ID NO: 61)
CᵢPYACᵢᵢTRLYGWCᵢᵢᵢ;
  (SEQ ID NO: 62)
CᵢSHACᵢᵢPRLTGWCᵢᵢᵢ;
  (SEQ ID NO: 63)
CᵢLHSCᵢᵢPRLSGWCᵢᵢᵢ;
  (SEQ ID NO: 64)
CᵢRHSCᵢᵢPILTGWCᵢᵢᵢ;
  (SEQ ID NO: 65)
CᵢGHSCᵢᵢPVLWGWCᵢᵢᵢ;
  (SEQ ID NO: 66)
CᵢPHSCᵢᵢPKLFGWCᵢᵢᵢ;
  (SEQ ID NO: 67)
CᵢTHSCᵢᵢPYLFGWCᵢᵢᵢ;
  (SEQ ID NO: 118)
CᵢDWTCᵢᵢYLTMMPCᵢᵢᵢ;
  (SEQ ID NO: 119)
CᵢDWTCᵢᵢYLRPLPCᵢᵢᵢ;
  (SEQ ID NO: 120)
CᵢDWTCᵢᵢYMSMKPCᵢᵢᵢ;
  (SEQ ID NO: 121)
CᵢDWTCᵢᵢYFRPLPCᵢᵢᵢ;
and
  (SEQ ID NO: 122)
CᵢDWTCᵢᵢYISPMFDCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 3 amino acids and the other of which consists of 6 amino acids and the bicyclic peptide ligand comprises an amino acid sequence which is selected from:

```
  (SEQ ID NO: 1)
CᵢHHACᵢᵢPILTGWCᵢᵢᵢ;
(SEQ ID NO: 6)
CᵢPHACᵢᵢPSLWGWCᵢᵢᵢ ;
  (SEQ ID NO: 7)
CᵢLHACᵢᵢPRLTHWCᵢᵢᵢ;
  (SEQ ID NO: 8)
CᵢLHACᵢᵢQYLWGYCᵢᵢᵢ;
  (SEQ ID NO: 9)
CᵢSHACᵢᵢPRLFGWCᵢᵢᵢ;
  (SEQ ID NO: 10)
CᵢQHACᵢᵢPYLWDYCᵢᵢᵢ;
  (SEQ ID NO: 58)
CᵢPFACᵢᵢHKLYGWCᵢᵢᵢ;
  (SEQ ID NO: 59)
CᵢMKACᵢᵢPYLYGWCᵢᵢᵢ;
  (SEQ ID NO: 60)
CᵢRHACᵢᵢTHLYGHCᵢᵢᵢ;
  (SEQ ID NO: 61)
CᵢPYACᵢᵢTRLYGWCᵢᵢᵢ;
  (SEQ ID NO: 62)
CᵢSHACᵢᵢPRLTGWCᵢᵢᵢ;
  (SEQ ID NO: 63)
CᵢLHSCᵢᵢPRLSGWCᵢᵢᵢ;
  (SEQ ID NO: 64)
CᵢRHSCᵢᵢPILTGWCᵢᵢᵢ;
(SEQ ID NO: 65)
CᵢGHSCᵢᵢPVLWGWCᵢᵢᵢ;
  (SEQ ID NO: 66)
CᵢPHSCᵢᵢPKLFGWCᵢᵢᵢ;
and
  (SEQ ID NO: 67)
CᵢTHSCᵢᵢPYLFGWCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 3 amino acids and the other of which consists of 6 amino acids, the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15230);
A-(SEQ ID NO: 6)-A (herein referred to as BCY15235);
A-(SEQ ID NO: 7)-A (herein referred to as BCY15236);
A-(SEQ ID NO: 8)-A (herein referred to as BCY15237);
A-(SEQ ID NO: 9)-A (herein referred to as BCY15238);
A-(SEQ ID NO: 10)-A (herein referred to as BCY15239);
A-(SEQ ID NO: 58)-A (herein referred to as BCY15364);
A-(SEQ ID NO: 59)-A (herein referred to as BCY15365);
A-(SEQ ID NO: 60)-A (herein referred to as BCY15366);
A-(SEQ ID NO: 61)-A (herein referred to as BCY15367);
A-(SEQ ID NO: 62)-A (herein referred to as BCY15368);
A-(SEQ ID NO: 63)-A (herein referred to as BCY15369);
A-(SEQ ID NO: 64)-A (herein referred to as BCY15370);
A-(SEQ ID NO: 65)-A (herein referred to as BCY15371);
A-(SEQ ID NO: 66)-A (herein referred to as BCY15372); and
A-(SEQ ID NO: 67)-A (herein referred to as BCY15373).

In a still yet further embodiment, said loop sequences comprise three reactive groups separated by two loop sequences one of which consists of 3 amino acids and the other of which consists of 6 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 6)-A-[Sar₆]-[KFl] (herein referred to as BCY15303); and
A-(SEQ ID NO: 63)-A-[Sar₆]-[KFl] (herein referred to as BCY15329).

In an alternative embodiment, said loop sequences comprise three reactive groups separated by two loop sequences one of which consists of 3 amino acids and the other of which consists of 6 amino acids, the molecular scaffold is TATB and the bicyclic peptide ligand additionally comprises N- and/or C-terminal additions and comprises an amino acid sequence which is selected from:
A-(SEQ ID NO: 118)-A (herein referred to as BCY15444);
A-(SEQ ID NO: 119)-A (herein referred to as BCY16927);
A-(SEQ ID NO: 120)-A (herein referred to as BCY16930);
A-(SEQ ID NO: 121)-A (herein referred to as BCY16933); and
A-(SEQ ID NO: 122)-A (herein referred to as BCY16940).

In an alternative embodiment, said loop sequences comprise three reactive groups separated by two loop sequences one of which consists of 3 amino acids and the other of which consists of 7 amino acids.

In a further embodiment, said loop sequences comprise three reactive groups separated by two loop sequences one of which consists of 3 amino acids and the other of which consists of 7 amino acids and the bicyclic peptide ligand comprises an amino acid sequence which is selected from:

```
  (SEQ ID NO: 123)
CᵢDWTCᵢᵢYLNIYHECᵢᵢᵢ;
  (SEQ ID NO: 124)
CᵢDWTCᵢᵢYMDYLSNCᵢᵢᵢ;
  (SEQ ID NO: 125)
CᵢDWTCᵢᵢYLRIHEACᵢᵢᵢ;
  (SEQ ID NO: 126)
CᵢDWTCᵢᵢYMRINDACᵢᵢᵢ;
and
  (SEQ ID NO: 127)
CᵢDWTCᵢᵢYINIYNTCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 3 amino acids and the other of which consists of 7 amino acids, the molecular scaffold is TATB and the bicyclic peptide ligand additionally comprises N- and/or C-terminal additions and comprises an amino acid sequence which is selected from:
A-(SEQ ID NO: 123)-A (herein referred to as BCY15445);
A-(SEQ ID NO: 124)-A (herein referred to as BCY16941);
A-(SEQ ID NO: 125)-A (herein referred to as BCY16942); and
A-(SEQ ID NO: 126)-A (herein referred to as BCY16946).

In an alternative embodiment, said loop sequences comprise three reactive groups separated by two loop sequences one of which consists of 3 amino acids and the other of which consists of 7 amino acids, the molecular scaffold is TCMT and the bicyclic peptide ligand additionally comprises N- and/or C-terminal additions and comprises an amino acid sequence which is:
A-(SEQ ID NO: 127)-A (herein referred to as BCY16948).

In an alternative embodiment, said loop sequences comprise three reactive groups separated by two loop sequences one of which consists of 4 amino acids and the other of which consists of 6 amino acids.

In a further embodiment, said loop sequences comprise three reactive groups separated by two loop sequences one of which consists of 4 amino acids and the other of which consists of 6 amino acids and the bicyclic peptide ligand comprises an amino acid sequence which is selected from:

```
  (SEQ ID NO: 29)
CᵢLTNDCᵢᵢHSDIRYCᵢᵢᵢ;
and
  (SEQ ID NO: 30)
CᵢITNDCᵢᵢHTSLIFCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 6 amino acids, the molecular scaffold is TCMT and the bicyclic peptide ligand additionally comprises N- and/or C-terminal additions and comprises an amino acid sequence which is selected from:
A-(SEQ ID NO: 29)-A (herein referred to as BCY15335); and
A-(SEQ ID NO: 30)-A (herein referred to as BCY15336).

In a still yet further embodiment, said loop sequences comprise three reactive groups separated by two loop sequences one of which consists of 4 amino acids and the other of which consists of 6 amino acids, the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 30)-A-[Sar₆]-[KFl] (herein referred to as BCY15314).

In an alternative embodiment, said loop sequences comprise three reactive groups separated by two loop sequences one of which consists of 4 amino acids and the other of which consists of 8 amino acids.

In a further embodiment, said loop sequences comprise three reactive groups separated by two loop sequences one of which consists of 4 amino acids and the other of which consists of 8 amino acids and the bicyclic peptide ligand comprises an amino acid sequence which is selected from:

```
  (SEQ ID NO: 3)
CᵢVDANCᵢᵢKIKILQRMCᵢᵢᵢ;
  (SEQ ID NO: 4)
CᵢTSSVCᵢᵢKIKELQRKCᵢᵢᵢ;
  (SEQ ID NO: 5)
CᵢRSLLCᵢᵢEYLQRTDSCᵢᵢᵢ;
  (SEQ ID NO: 14)
CᵢLTKSCᵢᵢKIKMLQRVCᵢᵢᵢ;
  (SEQ ID NO: 15)
CᵢMQPSCᵢᵢRVLQLQRVCᵢᵢᵢ;
  (SEQ ID NO: 16)
CᵢALPSCᵢᵢRILHLQHRCᵢᵢᵢ;
  (SEQ ID NO: 17)
CᵢHDAHCᵢᵢKILELQHRCᵢᵢᵢ;
  (SEQ ID NO: 18)
CᵢTSSHCᵢᵢRVLEEQRLCᵢᵢᵢ;
  (SEQ ID NO: 19)
CᵢPRDRCᵢᵢPTAWLYGLCᵢᵢᵢ;
  (SEQ ID NO: 20)
CᵢAEAGCᵢᵢRVKQLQQICᵢᵢᵢ;
  (SEQ ID NO: 21)
CᵢTPSPCᵢᵢRVKELQRACᵢᵢᵢ;
  (SEQ ID NO: 26)
CᵢSTANCᵢᵢRILELQQLCᵢᵢᵢ;
  (SEQ ID NO: 44)
CᵢVGRLCᵢᵢSTATDIRKCᵢᵢᵢ;
  (SEQ ID NO: 48 herein referred to as
BCY16983 when complexed with TATB)
CᵢRQSQCᵢᵢDWWAIRSFCᵢᵢᵢ;
  (SEQ ID NO: 49)
CᵢTDATCᵢᵢSIKRLQRLCᵢᵢᵢ;
  (SEQ ID NO: 50)
CᵢSPVSCᵢᵢPSGFKFGLCᵢᵢᵢ;
  (SEQ ID NO: 68)
CᵢDSPWCᵢᵢRIRSLQRQCᵢᵢᵢ;
  (SEQ ID NO: 69)
CᵢSVGACᵢᵢRVKLLQRVCᵢᵢᵢ;
  (SEQ ID NO: 70)
CᵢMFVPCᵢᵢAVREILGLCᵢᵢᵢ;
  (SEQ ID NO: 128)
CᵢSDLMCᵢᵢWYLQRTDSCᵢᵢᵢ;
  (SEQ ID NO: 129)
CᵢNSYMCᵢᵢWYLQRTDSCᵢᵢᵢ;
  (SEQ ID NO: 130)
CᵢTSYLCᵢᵢWYLQRTDSCᵢᵢᵢ;
and
  (SEQ ID NO: 131)
CᵢRSLMCᵢᵢWYLNQTDSCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 8 amino acids and the bicyclic peptide ligand comprises an amino acid sequence which is selected from:

```
  (SEQ ID NO: 3)
CᵢVDANCᵢᵢKIKILQRMCᵢᵢᵢ;
  (SEQ ID NO: 4)
CᵢTSSVCᵢᵢKIKELQRKCᵢᵢᵢ;
  (SEQ ID NO: 5)
CᵢRSLLCᵢᵢEYLQRTDSCᵢᵢᵢ;
  (SEQ ID NO: 14)
CᵢLTKSCᵢᵢKIKMLQRVCᵢᵢᵢ;
  (SEQ ID NO: 15)
CᵢMQPSCᵢᵢRVLQLQRVCᵢᵢᵢ;
  (SEQ ID NO: 16)
CᵢALPSCᵢᵢRILHLQHRCᵢᵢᵢ;
(SEQ ID NO: 17)
CᵢHDAHCᵢᵢKILELQHRCᵢᵢᵢ;
  (SEQ ID NO: 18)
CᵢTSSHCᵢᵢRVLEEQRLCᵢᵢᵢ;
  (SEQ ID NO: 19)
CᵢPRDRCᵢᵢPTAWLYGLCᵢᵢᵢ;
  (SEQ ID NO: 20)
CᵢAEAGCᵢᵢRVKQLQQICᵢᵢᵢ;
  (SEQ ID NO: 21)
CᵢTPSPCᵢᵢRVKELQRACᵢᵢᵢ;
  (SEQ ID NO: 26)
CᵢSTANCᵢᵢRILELQQLCᵢᵢᵢ;
  (SEQ ID NO: 44)
CᵢVGRLCᵢᵢSTATDIRKCᵢᵢᵢ;
  (SEQ ID NO: 48; herein referred to as
BCY16983 when complexed with TATB)
CᵢRQSQCᵢᵢDWWAIRSFCᵢᵢᵢ;
  (SEQ ID NO: 49)
CᵢTDATCᵢᵢSIKRLQRLCᵢᵢᵢ;
  (SEQ ID NO: 50)
CᵢSPVSCᵢᵢPSGFKFGLCᵢᵢᵢ;
  (SEQ ID NO: 68)
CᵢDSPWCᵢᵢRIRSLQRQCᵢᵢᵢ;
  (SEQ ID NO: 69)
CᵢSVGACᵢᵢRVKLLQRVCᵢᵢᵢ;
and
  (SEQ ID NO: 70)
CᵢMFVPCᵢᵢAVREILGLCᵢᵢᵢ;
```

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 8 amino acids, the molecular scaffold is TATB and the bicyclic peptide ligand additionally comprises N- and/or C-terminal additions and comprises an amino acid sequence which is selected from:
A-(SEQ ID NO: 3)-A (herein referred to as BCY15334);
A-(SEQ ID NO: 15)-A (herein referred to as BCY15244);
A-(SEQ ID NO: 16)-A (herein referred to as BCY15245);
A-(SEQ ID NO: 17)-A (herein referred to as BCY15246);
A-(SEQ ID NO: 18)-A (herein referred to as BCY15247);
A-(SEQ ID NO: 19)-A (herein referred to as BCY15248);
A-(SEQ ID NO: 20)-A (herein referred to as BCY15249);
A-(SEQ ID NO: 21)-A (herein referred to as BCY15250);
A-(SEQ ID NO: 26)-A (herein referred to as BCY15255);
A-(SEQ ID NO: 48)-A (herein referred to as BCY15354);
A-(SEQ ID NO: 48)-A (herein referred to as BCY16534);
A-(SEQ ID NO: 48)-AK (herein referred to as BCY16896);
A-(SEQ ID NO: 49)-A (herein referred to as BCY15355); and
A-(SEQ ID NO: 50)-A (herein referred to as BCY15356).

In a still yet further embodiment, said loop sequences comprise three reactive groups separated by two loop sequences one of which consists of 4 amino acids and the other of which consists of 8 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15301);
A-(SEQ ID NO: 15)-A-[Sar₆]-[KFl] (herein referred to as BCY15307);
A-(SEQ ID NO: 17)-A-[Sar₆]-[KFl] (herein referred to as BCY15308);
A-(SEQ ID NO: 19)-A-[Sar₆]-[KFl] (herein referred to as BCY15309);
A-(SEQ ID NO: 48)-A-[Sar₆]-[KFl] (herein referred to as BCY15324);
A-(SEQ ID NO: 49)-A-[Sar₆]-[KFl] (herein referred to as BCY15325); and
A-(SEQ ID NO: 50)-A-[Sar₆]-[KFl] (herein referred to as BCY15326).

In a yet further embodiment, said loop sequences comprise three reactive groups separated by two loop sequences one of which consists of 4 amino acids and the other of which consists of 8 amino acids, the molecular scaffold is TATA, the bicyclic peptide ligand additionally comprises N- and/or C-terminal additions and comprises an amino acid sequence which is selected from:
A-(SEQ ID NO: 3)-A (herein referred to as BCY15232);
A-(SEQ ID NO: 4)-A (herein referred to as BCY15233);
A-(SEQ ID NO: 5)-A (herein referred to as BCY15234);
A-(SEQ ID NO: 14)-A (herein referred to as BCY15243);
A-(SEQ ID NO: 44)-A (herein referred to as BCY15350);
A-(SEQ ID NO: 68)-A (herein referred to as BCY15374);
A-(SEQ ID NO: 69)-A (herein referred to as BCY15375);
A-(SEQ ID NO: 70)-A (herein referred to as BCY15376);
A-(SEQ ID NO: 128)-A (herein referred to as BCY16886);
A-(SEQ ID NO: 129)-A (herein referred to as BCY16887);
A-(SEQ ID NO: 130)-A (herein referred to as BCY16889); and
A-(SEQ ID NO: 131)-A (herein referred to as BCY16895).

In a still yet further embodiment, said loop sequences comprise three reactive groups separated by two loop sequences one of which consists of 4 amino acids and the other of which consists of 8 amino acids, the molecular scaffold is TATA, the bicyclic peptide ligand additionally comprises N- and/or C-terminal additions and comprises an amino acid sequence which is selected from:
A-(SEQ ID NO: 3)-A (herein referred to as BCY15232);
A-(SEQ ID NO: 4)-A (herein referred to as BCY15233);
A-(SEQ ID NO: 5)-A (herein referred to as BCY15234);
A-(SEQ ID NO: 14)-A (herein referred to as BCY15243);
A-(SEQ ID NO: 44)-A (herein referred to as BCY15350);
A-(SEQ ID NO: 68)-A (herein referred to as BCY15374);
A-(SEQ ID NO: 69)-A (herein referred to as BCY15375); and
A-(SEQ ID NO: 70)-A (herein referred to as BCY15376).

In a still yet further embodiment, said loop sequences comprise three reactive groups separated by two loop sequences one of which consists of 4 amino acids and the other of which consists of 8 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15300);
A-(SEQ ID NO: 5)-A-[Sar₆]-[KFl] (herein referred to as BCY15302); and
A-(SEQ ID NO: 70)-A-[Sar₆]-[KFl] (herein referred to as BCY15330).

In an alternative embodiment, said loop sequences comprise three reactive groups separated by two loop sequences one of which consists of 5 amino acids and the other of which consists of 3 amino acids.

In a further embodiment, said loop sequences comprise three reactive groups separated by two loop sequences one of which consists of 5 amino acids and the other of which consists of 3 amino acids and the bicyclic peptide ligand comprises an amino acid sequence which is:

(SEQ ID NO: 115)
CᵢFDDWTCᵢᵢYIQMCᵢᵢᵢ;

wherein Cᵢ, Cᵢᵢ and Cᵢᵢᵢ represent first, second and third cysteine residues, respectively, or a pharmaceutically acceptable salt thereof.

In a still yet further embodiment, said loop sequences comprise three reactive groups separated by two loop sequences one of which consists of 5 amino acids and the other of which consists of 3 amino acids, the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 115)-A-[Sar₆]-[KFl] (herein referred to as BCY15437).

In an alternative embodiment, said loop sequences comprise three reactive groups separated by two loop sequences one of which consists of 6 amino acids and the other of which consists of 3 amino acids.

In a further embodiment, said loop sequences comprise three reactive groups separated by two loop sequences one of which consists of 6 amino acids and the other of which consists of 3 amino acids and the bicyclic peptide ligand comprises an amino acid sequence which is selected from:

```
  (SEQ ID NO: 71)
CᵢTLMDPWCᵢᵢLLKCᵢᵢᵢ;
  (SEQ ID NO: 72)
CᵢKIHDWTCᵢᵢLLRCᵢᵢᵢ;
and
  (SEQ ID NO: 79; herein referred to as
BCY18707 when complexed with TATB)
CᵢIPLDWTCᵢᵢMIACᵢᵢᵢ;
```

wherein Cᵢ, Cᵢᵢ and Cᵢᵢᵢ represent first, second and third cysteine residues, respectively, or a pharmaceutically acceptable salt thereof.

In a further embodiment, said loop sequences comprise three reactive groups separated by two loop sequences one of which consists of 6 amino acids and the other of which consists of 3 amino acids and the bicyclic peptide ligand comprises an amino acid sequence which is selected from:

(SEQ ID NO: 71)
CᵢTLMDPWCᵢᵢLLKCᵢᵢᵢ;
(SEQ ID NO: 72)
CᵢKIHDWTCᵢᵢLLRCᵢᵢᵢ;
and
(SEQ ID NO: 79)
CᵢIPLDWTCᵢᵢMIACᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 3 amino acids, the molecular scaffold is TATA and the bicyclic peptide ligand additionally comprises N- and/or C-terminal additions and comprises an amino acid sequence which is selected from:
A-(SEQ ID NO: 71)-A (herein referred to as BCY15377); and
A-(SEQ ID NO: 72)-A (herein referred to as BCY15378).

In a still yet further embodiment, said loop sequences comprise three reactive groups separated by two loop sequences one of which consists of 6 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 71)-A-[Sar₆]-[KFl] (herein referred to as BCY15331).

In an alternative embodiment, said loop sequences comprise three reactive groups separated by two loop sequences one of which consists of 6 amino acids and the other of which consists of 3 amino acids, the molecular scaffold is TATB, the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446);
A-(SEQ ID NO: 79)-AK (herein referred to as BCY16994); and
Ac-(SEQ ID NO: 79)-K (herein referred to as BCY18654).

In a yet further alternative embodiment, said loop sequences comprise three reactive groups separated by two loop sequences one of which consists of 6 amino acids and the other of which consists of 3 amino acids, the molecular scaffold is TATB, the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446); and
A-(SEQ ID NO: 79)-AK (herein referred to as BCY16994).

In an alternative embodiment, said loop sequences comprise three reactive groups separated by two loop sequences one of which consists of 6 amino acids and the other of which consists of 4 amino acids.

In a further embodiment, said loop sequences comprise three reactive groups separated by two loop sequences one of which consists of 6 amino acids and the other of which consists of 4 amino acids and the bicyclic peptide ligand comprises an amino acid sequence which is selected from:

(SEQ ID NO: 11)
CᵢEYQGPHCᵢᵢYRLYCᵢᵢᵢ;
(SEQ ID NO: 132)
CᵢEYNGPYCᵢᵢYRLYCᵢᵢᵢ;
and
(SEQ ID NO: 133)
CᵢEYVGPMCᵢᵢYRLYCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 4 amino acids and the bicyclic peptide ligand comprises an amino acid sequence which is:

(SEQ ID NO: 11)
CᵢEYQGPHCᵢᵢYRLYCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 4 amino acids, the molecular scaffold is TATA, the bicyclic peptide ligand additionally comprises N- and/or C-terminal additions and comprises an amino acid sequence which is selected from:
A-(SEQ ID NO: 11)-A (herein referred to as BCY15240);
A-(SEQ ID NO: 132)-A (herein referred to as BCY17547); and
A-(SEQ ID NO: 133)-A (herein referred to as BCY17548).

In a still yet further embodiment, said loop sequences comprise three reactive groups separated by two loop sequences one of which consists of 6 amino acids and the other of which consists of 4 amino acids, the molecular scaffold is TATA, the bicyclic peptide ligand additionally comprises N- and/or C-terminal additions and comprises an amino acid sequence which is:

A-(SEQ ID NO: 11)-A (herein referred to as BCY15240).

In a still yet further embodiment, said loop sequences comprise three reactive groups separated by two loop sequences one of which consists of 6 amino acids and the other of which consists of 4 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 11)-A-[Sar₆]-[KFl] (herein referred to as BCY15304).

In an alternative embodiment, said loop sequences comprise three reactive groups separated by two loop sequences one of which consists of 7 amino acids and the other of which consists of 2 amino acids.

In a further embodiment, said loop sequences comprise three reactive groups separated by two loop sequences one of which consists of 7 amino acids and the other of which consists of 2 amino acids and the bicyclic peptide ligand comprises an amino acid sequence which is selected from:

(SEQ ID NO: 2)
CᵢEDHDWVYCᵢᵢSTCᵢᵢᵢ;
(SEQ ID NO: 23)
CHAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
(SEQ ID NO: 75)
CᵢPNISWIYCᵢᵢSTCᵢᵢᵢ;
(SEQ ID NO: 117)
CᵢDVCᵢᵢGLNAFNRCᵢᵢᵢ;
(SEQ ID NO: 134)
CᵢLDETWIYCᵢᵢSTCᵢᵢᵢ;
(SEQ ID NO: 135)
CᵢPDETWVYCᵢᵢSTCᵢᵢᵢ;
(SEQ ID NO: 136)
CᵢESNDWVYCᵢᵢSTCᵢᵢᵢ;
(SEQ ID NO: 137)
CᵢEDNDWVYCᵢᵢSTCᵢᵢᵢ;
(SEQ ID NO: 138)
CᵢPDVSWIYCᵢᵢSTCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 2 amino acids and the bicyclic peptide ligand comprises an amino acid sequence which is selected from:

(SEQ ID NO: 2)
CᵢEDHDWVYCᵢᵢSTCᵢᵢᵢ;
(SEQ ID NO: 23)
CᵢAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
(SEQ ID NO: 75)
CᵢPNISWIYCᵢᵢSTCᵢᵢᵢ;
and
(SEQ ID NO: 117)
CᵢDVCᵢᵢGLNAFNRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 2 amino acids and the bicyclic peptide ligand comprises an amino acid sequence which is selected from:

(SEQ ID NO: 2)
CᵢEDHDWVYCᵢᵢSTCᵢᵢᵢ;
(SEQ ID NO: 23)
CᵢAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
and
(SEQ ID NO: 75)
CᵢPNISWIYCᵢᵢSTCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380);
A-(SEQ ID NO: 75)-A (herein referred to as BCY15381);
A-(SEQ ID NO: 134)-A (herein referred to as BCY17540);
A-(SEQ ID NO: 135)-A (herein referred to as BCY17541);
A-(SEQ ID NO: 136)-A (herein referred to as BCY17542);
A-(SEQ ID NO: 137)-A (herein referred to as BCY17543); and
A-(SEQ ID NO: 138)-A (herein referred to as BCY17544).

In a still yet further embodiment, said loop sequences comprise three reactive groups separated by two loop sequences one of which consists of 7 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380); and
A-(SEQ ID NO: 75)-A (herein referred to as BCY15381).

In a still yet further embodiment, said loop sequences comprise three reactive groups separated by two loop sequences one of which consists of 7 amino acids and the other of which consists of 2 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 2)-A-[Sar₆]-[KFl] (herein referred to as BCY15299); and
A-(SEQ ID NO: 74)-A-[Sar₆]-[KFl] (herein referred to as BCY15332).

In a still yet further embodiment, said loop sequences comprise three reactive groups separated by two loop sequences one of which consists of 7 amino acids and the other of which consists of 2 amino acids, the molecular scaffold is TCMT, the bicyclic peptide additionally comprises N- and/or C-terminal additions and a labelling moiety, such as fluorescein (Fl), and comprises an amino acid sequence which is:

A-(SEQ ID NO: 117)-A-[Sar₆]-[KFl] (herein referred to as BCY16287).

In a yet further embodiment, said loop sequences comprise three reactive groups separated by two loop sequences one of which consists of 7 amino acids and the other of which consists of 2 amino acids, the molecular scaffold is TATB, the bicyclic peptide ligand additionally comprises N- and/or C-terminal additions and comprises an amino acid sequence which is selected from:
A-(SEQ ID NO: 23)-A (herein referred to as BCY15252);
A-(SEQ ID NO: 25)-A (herein referred to as BCY15254);
A-(SEQ ID NO: 28)-A (herein referred to as BCY15257);
A-(SEQ ID NO: 51)-A (herein referred to as BCY15357);
A-(SEQ ID NO: 52)-A (herein referred to as BCY15358);
A-(SEQ ID NO: 53)-A (herein referred to as BCY15359);
A-(SEQ ID NO: 54)-A (herein referred to as BCY15360); and
A-(SEQ ID NO: 55)-A (herein referred to as BCY15361).

In a still yet further embodiment, said loop sequences comprise three reactive groups separated by two loop sequences one of which consists of 7 amino acids and the other of which consists of 2 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 23)-A-[Sar₆]-[KFl] (herein referred to as BCY15311);
A-(SEQ ID NO: 25)-A-[Sar₆]-[KFl] (herein referred to as BCY15312); and
A-(SEQ ID NO: 53)-A-[Sar₆]-[KFl] (herein referred to as BCY15327).

In an alternative embodiment, said loop sequences comprise three reactive groups separated by two loop sequences one of which consists of 7 amino acids and the other of which consists of 3 amino acids.

In a further embodiment, said loop sequences comprise three reactive groups separated by two loop sequences one of which consists of 7 amino acids and the other of which consists of 3 amino acids and the bicyclic peptide ligand comprises an amino acid sequence which is selected from:

(SEQ ID NO: 22; herein referred to as
BCY16534 when complexed with TATB)
CᵢASPDNPVCᵢᵢRFYC_{iii;}
(SEQ ID NO: 24; herein referred to as
BCY16540 when complexed with TATB)
CᵢYNHANPVCᵢᵢRYYC_{iii;}
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
(SEQ ID NO: 114)
CᵢYNHANPVCᵢᵢ[Arg(Me)]YYCᵢᵢᵢ;
(SEQ ID NO: 116)
CᵢMNPFFYDCᵢᵢERTCᵢᵢᵢ;
(SEQ ID NO: 139)
Cᵢ[Aib]SPDNPVCᵢᵢRFYCᵢᵢᵢ;
(SEQ ID NO: 140)
CᵢAS[HyP]DNPVCᵢᵢRFYCᵢᵢᵢ;
(SEQ ID NO: 141)
CᵢAS[Aib]DNPVCᵢᵢRFYCᵢᵢᵢ;
(SEQ ID NO: 142)
CᵢAS[Pip]DNPVCᵢᵢRFYCᵢᵢᵢ;
(SEQ ID NO: 143)
CᵢASPDN[Pip]VCᵢᵢRFYCᵢᵢᵢ;
(SEQ ID NO: 144)
CᵢASPDN[44DFP]VCᵢᵢRFYCᵢᵢᵢ;
(SEQ ID NO: 145)
CᵢASPDN[4FlPro]VCᵢᵢRFYCᵢᵢᵢ;
(SEQ ID NO: 146)
CᵢASPDNPVCᵢᵢR[1Nal]YCᵢᵢᵢ;
(SEQ ID NO: 147)
CᵢASPDNPVCᵢᵢR[2Nal]YCᵢᵢᵢ;
(SEQ ID NO: 148)
CᵢASPDNPVCᵢᵢR[2MePhe]YCᵢᵢᵢ;
(SEQ ID NO: 149)
CᵢASPDNPVCᵢᵢR[3MePhe]YCᵢᵢᵢ;
(SEQ ID NO: 150)
CᵢASPDNPVCᵢᵢR[4MePhe]YCᵢᵢᵢ;
(SEQ ID NO: 151)
CᵢASPDNPVCᵢᵢR[2ClPhe]YCᵢᵢᵢ;
(SEQ ID NO: 152)
CᵢASPDNPVCᵢᵢR[3ClPhe]YCᵢᵢᵢ;
(SEQ ID NO: 153)
CᵢASPDNPVCᵢᵢR[4ClPhe]YCᵢᵢᵢ;
(SEQ ID NO: 154)
CᵢASPDNPVCᵢᵢR[2FPhe]YCᵢᵢᵢ;
(SEQ ID NO: 155)
CᵢASPDNPVCᵢᵢR[3FPhe]YCᵢᵢᵢ;
(SEQ ID NO: 156)
CᵢASPDNPVCᵢᵢR[4FPhe]YCᵢᵢᵢ;
(SEQ ID NO: 157)
CᵢASPDNPVCᵢᵢR[26DiMeTyr]YCᵢᵢᵢ;
(SEQ ID NO: 158)
Cᵢ[Aib]SPDN[44DFP]VCᵢᵢR[4FPhe]YCᵢᵢᵢ;
(SEQ ID NO: 159)
Cᵢ[Aib]SPDN[44DFP]VCᵢᵢ[Arg(Me)][4FPhe]YCᵢᵢᵢ;
(SEQ ID NO: 160)
Cᵢ[Aib]SPDNPVCᵢᵢR[4FPhe]YCᵢᵢᵢ;
(SEQ ID NO: 161)
Cᵢ[Aib]SPDNPVCᵢᵢ[Arg(Me)][4FPhe]YCᵢᵢᵢ;
(SEQ ID NO: 162)
Cᵢ[Aib]NPDN[44DFP]VCᵢᵢR[4FPhe]YCᵢᵢᵢ;
(SEQ ID NO: 163)
Cᵢ[Aib]NPDN[44DFP]VCᵢᵢ[Arg(Me)][4FPhe]YCᵢᵢᵢ;
(SEQ ID NO: 164)
Cᵢ[Aib]NPDNPVCᵢᵢR[4FPhe]YCᵢᵢᵢ;
and
(SEQ ID NO: 165)
Cᵢ[Aib]NPDNPVCᵢᵢ[Arg(Me)][4FPhe]YCᵢᵢᵢ;

wherein Cᵢ, Cᵢᵢ and Cᵢᵢᵢ represent first, second and third cysteine residues, respectively, Aib represents aminoisobutyric acid, Agb represents 2-amino-4-guanidinobutyric acid, Arg(Me) represents δ-N methyl arginine, 2ClPhe represents 2-chloro-phenylalanine, 3ClPhe represents 3-chloro-phenylalanine, 4ClPhe represents 4-chloro-phenylalanine, 44DFP represents 4,4-difluoroproline, 26DiMeTyr represents 2,6-dimethyl-tyrosine, 2FPhe represents 2-fluoro-phenylalanine, 3FPhe represents 3-fluoro-phenylalanine, 4FPhe represents 4-fluoro-phenylalanine, 4FlPro represents 4-fluoro-proline, HArg represents homoarginine, HyP represents hydroxyproline, 2MePhe represents 2-methyl-phenylalanine, 3MePhe represents 3-methyl-phenylalanine, 4MePhe represents 4-methyl-phenylalanine, 1Nal represents 1-naphthylalanine, 2Nal represents 2-naphthylalanine, Pip represents pipecolic acid, or a pharmaceutically acceptable salt thereof.

In a yet further embodiment, said loop sequences comprise three reactive groups separated by two loop sequences one of which consists of 7 amino acids and the other of which consists of 3 amino acids and the bicyclic peptide ligand comprises an amino acid sequence which is selected from:

(SEQ ID NO: 22; herein referred to as
BCY16534 when complexed with TATB)
CᵢASPDNPVCᵢᵢRFYC_{iii;}
(SEQ ID NO: 24; herein referred to as
BCY16540 when complexed with TATB)
CᵢYNHANPVCᵢᵢRYYC_{iii;}
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CᵢHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢCASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
(SEQ ID NO: 114)
CᵢYNHANPVCᵢᵢ[Arg(Me)]YYCᵢᵢᵢ;
and
(SEQ ID NO: 116)
CᵢMNPFFYDCᵢᵢERTCᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents 8-N methyl arginine, HArg represents homoarginine, or a pharmaceutically acceptable salt thereof.

In a yet further embodiment, said loop sequences comprise three reactive groups separated by two loop sequences one of which consists of 7 amino acids and the other of which consists of 3 amino acids and the bicyclic peptide ligand comprises an amino acid sequence which is selected from:

(SEQ ID NO: 22; herein referred to
as BCY16534 when complexed with TATB)
CᵢASPDNPVCᵢᵢRFYCᵢᵢᵢ;
(SEQ ID NO: 24; herein referred to as
BCY16540 when complexed with TATB)
CᵢYNHANPVCᵢᵢRYYC_{iii;}
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CᵢHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢCASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
and
(SEQ ID NO: 114)
CᵢYNHANPVCᵢᵢ[Arg(Me)]YYCᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents δ-N methyl arginine, HArg represents homoarginine, or a pharmaceutically acceptable salt thereof.

In a yet further embodiment, said loop sequences comprise three reactive groups separated by two loop sequences one of which consists of 7 amino acids and the other of which consists of 3 amino acids, the molecular scaffold is TATB, the bicyclic peptide ligand additionally comprises N- and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543);
A-(SEQ ID NO: 114)-A (herein referred to as BCY16542);
A-(SEQ ID NO: 116)-A (herein referred to as BCY16207);
Ac-(SEQ ID NO: 116) (herein referred to as BCY18698);
Ac-(SEQ ID NO: 139) (herein referred to as BCY17279);
Ac-(SEQ ID NO: 140) (herein referred to as BCY17281);
Ac-(SEQ ID NO: 141) (herein referred to as BCY17282);
Ac-(SEQ ID NO: 142) (herein referred to as BCY17283);
Ac-(SEQ ID NO: 143) (herein referred to as BCY17287);
Ac-(SEQ ID NO: 144) (herein referred to as BCY17289);
Ac-(SEQ ID NO: 145) (herein referred to as BCY17294);
Ac-(SEQ ID NO: 146) (herein referred to as BCY17301);
Ac-(SEQ ID NO: 147) (herein referred to as BCY17302);
Ac-(SEQ ID NO: 148) (herein referred to as BCY17303);
Ac-(SEQ ID NO: 149) (herein referred to as BCY17304);
Ac-(SEQ ID NO: 150) (herein referred to as BCY17305);
Ac-(SEQ ID NO: 151) (herein referred to as BCY17306);
Ac-(SEQ ID NO: 152) (herein referred to as BCY17307);
Ac-(SEQ ID NO: 153) (herein referred to as BCY17308);
Ac-(SEQ ID NO: 154) (herein referred to as BCY17309);
Ac-(SEQ ID NO: 155) (herein referred to as BCY17310);
Ac-(SEQ ID NO: 156) (herein referred to as BCY17311);
Ac-(SEQ ID NO: 157) (herein referred to as BCY17313);
Ac-(SEQ ID NO: 158) (herein referred to as BCY18340);
Ac-(SEQ ID NO: 159) (herein referred to as BCY18341);
Ac-(SEQ ID NO: 160) (herein referred to as BCY18342);
Ac-(SEQ ID NO: 161) (herein referred to as BCY18343);
Ac-(SEQ ID NO: 162) (herein referred to as BCY18344);
Ac-(SEQ ID NO: 163) (herein referred to as BCY18345);
Ac-(SEQ ID NO: 164) (herein referred to as BCY18346); and
Ac-(SEQ ID NO: 165) (herein referred to as BCY18347).

In a still yet further embodiment, said loop sequences comprise three reactive groups separated by two loop sequences one of which consists of 7 amino acids and the other of which consists of 3 amino acids, the molecular scaffold is TATB, the bicyclic peptide ligand additionally comprises N- and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543); and
A-(SEQ ID NO: 114)-A (herein referred to as BCY16542).

In a still yet further embodiment, said loop sequences comprise three reactive groups separated by two loop sequences one of which consists of 7 amino acids and the other of which consists of 3 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 22)-A-[Sar₆]-[KFl] (herein referred to as BCY15310);
A-(SEQ ID NO: 27)-A-[Sar₆]-[KFl] (herein referred to as BCY15313);
A-(SEQ ID NO: 56)-A-[Sar₆]-[KFl] (herein referred to as BCY15328); and
A-(SEQ ID NO: 116)-A-[Sar₆]-[KFl] (herein referred to as BCY16298).

In a still yet further embodiment, said loop sequences comprise three reactive groups separated by two loop sequences one of which consists of 7 amino acids and the other of which consists of 3 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 22)-A-[Sar₆]-[KFl] (herein referred to as BCY15310);
A-(SEQ ID NO: 27)-A-[Sar₆]-[KFl] (herein referred to as BCY15313); and
A-(SEQ ID NO: 56)-A-[Sar₆]-[KFl] (herein referred to as BCY15328).

In a yet further embodiment, said loop sequences comprise three reactive groups separated by two loop sequences one of which consists of 7 amino acids and the other of which consists of 3 amino acids, the molecular scaffold is TCMT, the bicyclic peptide ligand additionally comprises N- and/or C-terminal additions and comprises an amino acid sequence which is:
A-(SEQ ID NO: 31)-A (herein referred to as BCY15315).

In a still yet further embodiment, said loop sequences comprise three reactive groups separated by two loop sequences one of which consists of 7 amino acids and the other of which consists of 3 amino acids, the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 31)-A-[Sar₆]-[KFl] (herein referred to as BCY15313).

In a yet further embodiment, said loop sequences comprise three reactive groups separated by two loop sequences one of which consists of 7 amino acids and the other of which consists of 3 amino acids, the molecular scaffold is TATA, the bicyclic peptide ligand additionally comprises N- and/or C-terminal additions and comprises an amino acid sequence which is:
A-(SEQ ID NO: 76)-A (herein referred to as BCY15382).

In a still yet further embodiment, said loop sequences comprise three reactive groups separated by two loop sequences one of which consists of 7 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 76)-A-[Sar₆]-[KFl] (herein referred to as BCY15333).

In an alternative embodiment, said loop sequences comprise three reactive groups separated by two loop sequences one of which consists of 7 amino acids and the other of which consists of 5 amino acids.

In a further embodiment, said loop sequences comprise three reactive groups separated by two loop sequences one of which consists of 7 amino acids and the other of which consists of 5 amino acids and the bicyclic peptide ligand comprises an amino acid sequence which is selected from:

(SEQ ID NO: 32)
CᵢTTSEKVKCᵢᵢLQRHPCᵢᵢᵢ;
(SEQ ID NO: 33)
CᵢQPDMRIKCᵢᵢLQRVACᵢᵢᵢ;
(SEQ ID NO: 34)
CᵢSSNNRIKCᵢᵢLQRVTCᵢᵢᵢ;
(SEQ ID NO: 35)
CᵢKEKTTIGCᵢᵢLMAGICᵢᵢᵢ;
and
(SEQ ID NO: 166)
CᵢNRPTSVYCᵢᵢLQRGICᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 5 amino acids and the bicyclic peptide ligand comprises an amino acid sequence which is selected from:

(SEQ ID NO: 32)
CᵢTTSEKVKCᵢᵢLQRHPCᵢᵢᵢ;
(SEQ ID NO: 33)
CᵢQPDMRIKCᵢᵢLQRVACᵢᵢᵢ;
(SEQ ID NO: 34)
CᵢSSNNRIKCᵢᵢLQRVTCᵢᵢᵢ;
and
(SEQ ID NO: 35)
CᵢKEKTTIGCᵢᵢLMAGICᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 5 amino acids, the molecular scaffold is TCMT, the bicyclic peptide ligand additionally comprises N- and/or C-terminal additions and comprises an amino acid sequence which is selected from:
A-(SEQ ID NO: 32)-A (herein referred to as BCY15338);
A-(SEQ ID NO: 33)-A (herein referred to as BCY15339);
A-(SEQ ID NO: 34)-A (herein referred to as BCY15340);
A-(SEQ ID NO: 35)-A (herein referred to as BCY15341); and
A-(SEQ ID NO: 166)-A (herein referred to as BCY17359).

In a still yet further embodiment, said loop sequences comprise three reactive groups separated by two loop sequences one of which consists of 7 amino acids and the other of which consists of 5 amino acids, the molecular scaffold is TCMT, the bicyclic peptide ligand additionally comprises N- and/or C-terminal additions and comprises an amino acid sequence which is selected from:
A-(SEQ ID NO: 32)-A (herein referred to as BCY15338);
A-(SEQ ID NO: 33)-A (herein referred to as BCY15339);
A-(SEQ ID NO: 34)-A (herein referred to as BCY15340); and
A-(SEQ ID NO: 35)-A (herein referred to as BCY15341).

In a still yet further embodiment, said loop sequences comprise three reactive groups separated by two loop sequences one of which consists of 7 amino acids and the other of which consists of 5 amino acids, the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 32)-A-[Sar₆]-[KFl] (herein referred to as BCY15316); and
A-(SEQ ID NO: 33)-A-[Sar₆]-[KFl] (herein referred to as BCY15317).

In an alternative embodiment, said loop sequences comprise three reactive groups separated by two loop sequences one of which consists of 8 amino acids and the other of which consists of 2 amino acids.

In a further embodiment, said loop sequences comprise three reactive groups separated by two loop sequences one of which consists of 8 amino acids and the other of which consists of 2 amino acids and the bicyclic peptide ligand comprises an amino acid sequence which is selected from:

(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as
BCY16312 when complexed with TCMT)
CᵢPYVAGRGTCᵢᵢLLCᵢᵢᵢ,
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
(SEQ ID NO: 88)
CᵢPYVAG[Agb][dA]TCᵢᵢL[tBuAla]Cᵢᵢᵢ;
(SEQ ID NO: 167; herein referred to as
BCY18111 when complexed with TCMT)
CᵢPYVAG[HArg][dA]TCᵢᵢL[tBuAla]Cᵢᵢᵢ;
(SEQ ID NO: 168)
CᵢPYVPG[HArg][dA]TCᵢᵢL[tBuAla]Cᵢᵢᵢ;
(SEQ ID NO: 169)
Cᵢ[K(PYA)]YVAG[HArg][dA]TCᵢᵢL[tBuAla]Cᵢᵢᵢ;
(SEQ ID NO: 170)
Cᵢ[HyP]YVAG[HArg][dA]TCᵢᵢL[tBuAla]Cᵢᵢᵢ;
(SEQ ID NO: 171)
CᵢPYVAGT[dA]TCᵢᵢL[tBuAla]Cᵢᵢᵢ;
(SEQ ID NO: 172)
CᵢPYVAG[Agb][dA]TCᵢᵢLLCᵢᵢᵢ;
(SEQ ID NO: 173)
CᵢPYVAG[Agb]GTCᵢᵢL[tBuAla]Cᵢᵢᵢ;
(SEQ ID NO: 174)
CᵢPYV[HyP]G[HArg][dA]TCᵢᵢL[tBuAla]Cᵢᵢᵢ;
(SEQ ID NO: 175)
CᵢPY[K(PYA)]AG[HArg][dA]TCᵢᵢL[tBuAla]Cᵢᵢᵢ;
(SEQ ID NO: 176)
Cᵢ[Oic]YVAG[HArg][dA]TCᵢᵢL[tBuAla]Cᵢᵢᵢ;
(SEQ ID NO: 177)
CᵢPY[B-Melle]AG[HArg][dA]TCᵢᵢL[tBuAla]Cᵢᵢᵢ;
(SEQ ID NO: 178)
CᵢPY[tBuGly]AG[HArg][dA]TCᵢL[tBuAla]Cᵢᵢᵢ;
(SEQ ID NO: 179)
CᵢPYPAG[HArg][dA]TCᵢᵢL[tBuAla]Cᵢ;
(SEQ ID NO: 180)
CᵢP[44BPA]VAG[HArg][dA]TCᵢᵢL[tBuAla]Cᵢᵢᵢ;
(SEQ ID NO: 181)
C;P[2FPhe]VAG[HArg][dA]TCᵢᵢL[tBuAla]Cᵢᵢᵢ;
(SEQ ID NO: 182)
CᵢPY[Cba]AG[HArg][dA]TCᵢᵢL[tBuAla]Cᵢᵢᵢ;
(SEQ ID NO: 183)
CᵢP[3FPhe]VAG[HArg][dA]TCᵢᵢL[tBuAla]Cᵢᵢᵢ;
(SEQ ID NO: 184)
Cᵢ[55DMP]YVAG[HArg][dA]TCᵢᵢL[tBuAla]Cᵢ;
(SEQ ID NO: 185)
CᵢPYVAGQ[dA]TCᵢᵢL[tBuAla]Cᵢᵢᵢ;
(SEQ ID NO: 186)
CᵢPYVAG[HArg][dS]TCᵢᵢL[tBuAla]Cᵢᵢᵢ;
(SEQ ID NO: 187)
CᵢP[4tBuPhe]VAG[HArg][dA]TCᵢᵢL[tBuAla]Cᵢᵢᵢ;
(SEQ ID NO: 188)
CᵢPYREGTGTCᵢᵢLLCᵢᵢᵢ;
(SEQ ID NO: 189)
CᵢPYAPGNGTCᵢᵢLLCᵢᵢᵢ;
(SEQ ID NO: 190)
CᵢPHPPGRGTCᵢᵢLLCᵢᵢᵢ;
(SEQ ID NO: 191)
CᵢPYNAGTGTCᵢᵢLLCᵢᵢᵢ;
(SEQ ID NO: 192)
CᵢPYSPGQGTCᵢᵢLLCᵢᵢᵢ;
(SEQ ID NO: 193)
CᵢPYQPGSGTCᵢᵢLLCᵢᵢᵢ;
(SEQ ID NO: 194)
CᵢPFPPGMGTCᵢᵢLLCᵢᵢᵢ;
(SEQ ID NO: 195)
CᵢPHQPGFGTCᵢᵢLLCᵢᵢᵢ;
(SEQ ID NO: 196)
CᵢPYSPGSGTCᵢᵢLLCᵢᵢᵢ;
(SEQ ID NO: 197)
CᵢPYLAGTGTCᵢᵢLLCᵢᵢᵢ;
(SEQ ID NO: 198)
CᵢPWEAGKGTCᵢᵢLLCᵢᵢᵢ;
(SEQ ID NO: 199)
CᵢPYAPGMGTCᵢᵢLLCᵢᵢᵢ;
(SEQ ID NO: 200)
CᵢPHMPGSGTCᵢᵢLLCᵢᵢᵢ;
(SEQ ID NO: 201)
CᵢPYNKGEGTCᵢᵢLLCᵢᵢᵢ;
(SEQ ID NO: 202)
CᵢPFKPGVGTCᵢᵢLLCᵢᵢᵢ;
(SEQ ID NO: 203)
CᵢP[4tBuPhe]VAG[Orn][dA]TCᵢᵢL[tBuAla]Cᵢᵢᵢ;
(SEQ ID NO: 204)
Cᵢ[Oic][4tBuPhe]VAG[HArg][dA]TCᵢL[tBuAla]Cᵢᵢᵢ;
(SEQ ID NO: 205)
CᵢP[4tBuPhe]VAG[Dab][dA]TCᵢᵢL[tBuAla]Cᵢᵢᵢ;
(SEQ ID NO: 206)
CᵢP[4tBuPhe]VAG[Dap][dA]TCᵢᵢL[tBuAla]Cᵢᵢᵢ;
(SEQ ID NO: 207)
CᵢP[4CF3Phe]VAG[HArg][dA]TCᵢᵢL[tBuAla]Cᵢᵢᵢ;
(SEQ ID NO: 208)
CᵢP[DMAPhe]VAG[HArg][dA]TCᵢL[tBuAla]Cᵢᵢᵢ;
(SEQ ID NO: 209)
CᵢP[4tBuPhe]VAG[HArg][dDab]TCᵢᵢL[tBuAla]Cᵢᵢᵢ;
(SEQ ID NO: 210)
C;P[4tBuPhe]VAG[HSer][dA]TCᵢᵢL[tBuAla]Cᵢᵢᵢ;
(SEQ ID NO: 211)
CᵢP[4tBuPhe]VAG[Cᵢt][dA]TCᵢᵢL[tBuAla]Cᵢᵢᵢ;
(SEQ ID NO: 212)
C[Oic][4tBuPhe]VAG[Orn][dA]TCᵢᵢL[tBuAla]Cᵢᵢᵢ;
(SEQ ID NO: 213)
CᵢP[4tBuPhe]VAG[HArg][dDap]TCᵢᵢL[tBuAla]Cᵢᵢᵢ;
and
(SEQ ID NO: 214)
Cᵢ[Oic][4tBuPhe]VAG[Cᵢt][dA]TCᵢL[tBuAla]Cᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, B-Melle represents β-methyl-isoleucine, 44BPA represents 4,4-biphenylalanine, Cba represents β-cyclobutylalanine, 4CF3Phe represents 4-trifluoromethyl-phenylalanine, Cit represents citrulline, Dab represents diaminobutanoic acid, Dap represents diaminopropionic acid, DMAPhe represents 4-Dimethylamino-phenylalanine, 55DMP represents 5,5-Dimethyl-L-proline, 2FPhe represents 2-fluoro-phenylalanine, 3FPhe represents 3-fluoro-phenylalanine, HArg represents homoarginine, HSer represents homoserine, HyP represents hydroxyproline, Oic represents octahydroindolecarboxylic acid, Orn represents ornithine, PYA represents pentynoic acid, tBuAla represents t-butyl-alanine, tBuGly represents t-butyl-glycine, 4tBuPhe represents 4-t-butyl-phenylalanine, or a pharmaceutically acceptable salt thereof.

In a yet further embodiment, said loop sequences comprise three reactive groups separated by two loop sequences one of which consists of 8 amino acids and the other of which consists of 2 amino acids and the bicyclic peptide ligand comprises an amino acid sequence which is selected from:

(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as
BCY16312 when complexed with TCMT)
CᵢPYVAGRGTCᵢᵢLLCᵢᵢᵢ;
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
and
(SEQ ID NO: 88)
CᵢPYVAG[Agb][dA]TCᵢᵢL[tBuAla]Cᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents δ-N methyl arginine, Cba represents β-cyclobutylalanine, HArg represents homoarginine, tBuAla represents t-butyl-alanine, or a pharmaceutically acceptable salt thereof.

In a yet further embodiment, said loop sequences comprise three reactive groups separated by two loop sequences one of which consists of 8 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 12)-A (herein referred to as BCY15241);
A-(SEQ ID NO: 13)-A (herein referred to as BCY15242);
A-(SEQ ID NO: 77)-A (herein referred to as BCY15383); and
A-(SEQ ID NO: 78)-A (herein referred to as BCY15384).

In a still yet further embodiment, said loop sequences comprise three reactive groups separated by two loop sequences one of which consists of 8 amino acids and the other of which consists of 2 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 12)-A-[Sar₆]-[KFl] (herein referred to as BCY15305); and
A-(SEQ ID NO: 13)-A-[Sar₆]-[KFl] (herein referred to as BCY15306.

In a yet further embodiment, said loop sequences comprise three reactive groups separated by two loop sequences one of which consists of 8 amino acids and the other of which consists of 2 amino acids, the molecular scaffold is TCMT, the bicyclic peptide ligand additionally comprises N- and/or C-terminal additions and comprises an amino acid sequence which is selected from:
A-(SEQ ID NO: 36)-A (herein referred to as BCY15342);
A-(SEQ ID NO: 37)-A (herein referred to as BCY15343);
Ac-A-(SEQ ID NO: 37)-A (herein referred to as BCY16322);
Ac-(SEQ ID NO: 37) (herein referred to as BCY16926);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
Ac-(SEQ ID NO: 80) (herein referred to as BCY18086);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321);
Ac-(SEQ ID NO: 88) (herein referred to as BCY16591);
Ac-(SEQ ID NO: 167) (herein referred to as BCY18024);
Ac-(SEQ ID NO: 168)-[K(PYA)] (herein referred to as BCY18025);
Ac-(SEQ ID NO: 169) (herein referred to as BCY18026);
Ac-(SEQ ID NO: 170)-[K(PYA)] (herein referred to as BCY18027);
Ac-(SEQ ID NO: 171)-[K(PYA)] (herein referred to as BCY18040);
Ac-(SEQ ID NO: 172) (herein referred to as BCY18087);
Ac-(SEQ ID NO: 173) (herein referred to as BCY18088);
Ac-(SEQ ID NO: 174)-[K(PYA)] (herein referred to as BCY18109);
Ac-(SEQ ID NO: 175) (herein referred to as BCY18110);
Ac-(SEQ ID NO: 176)-[K(PYA)] (herein referred to as BCY18115);
Ac-(SEQ ID NO: 177)-[K(PYA)] (herein referred to as BCY18211);
Ac-(SEQ ID NO: 178)-[K(PYA)] (herein referred to as BCY18212);
Ac-(SEQ ID NO: 179)-[K(PYA)] (herein referred to as BCY18351);
Ac-(SEQ ID NO: 180)-[K(PYA)] (herein referred to as BCY18524);
Ac-(SEQ ID NO: 181)-[K(PYA)] (herein referred to as BCY18527);
Ac-(SEQ ID NO: 182)-[K(PYA)] (herein referred to as BCY18529);
Ac-(SEQ ID NO: 183)-[K(PYA)] (herein referred to as BCY18661);
Ac-(SEQ ID NO: 184)-[K(PYA)] (herein referred to as BCY18662);
Ac-(SEQ ID NO: 185) (herein referred to as BCY19305);
Ac-(SEQ ID NO: 186) (herein referred to as BCY19309);
Ac-(SEQ ID NO: 187)-[K(PYA)] (herein referred to as BCY19378);
Ac-(SEQ ID NO: 188) (herein referred to as BCY19533);
Ac-(SEQ ID NO: 189) (herein referred to as BCY19534);
Ac-(SEQ ID NO: 190) (herein referred to as BCY19535);
Ac-(SEQ ID NO: 191) (herein referred to as BCY19536);
Ac-(SEQ ID NO: 192) (herein referred to as BCY19537);
Ac-(SEQ ID NO: 193) (herein referred to as BCY19538);
Ac-(SEQ ID NO: 194) (herein referred to as BCY19539);
Ac-(SEQ ID NO: 195) (herein referred to as BCY19541);
Ac-(SEQ ID NO: 196) (herein referred to as BCY19542);
Ac-(SEQ ID NO: 197) (herein referred to as BCY19543);
Ac-(SEQ ID NO: 198) (herein referred to as BCY19544);
Ac-(SEQ ID NO: 199) (herein referred to as BCY19545);
Ac-(SEQ ID NO: 200) (herein referred to as BCY19546);
Ac-(SEQ ID NO: 201) (herein referred to as BCY19547);
Ac-(SEQ ID NO: 202) (herein referred to as BCY19548);
Ac-(SEQ ID NO: 203)-[K(PYA)] (herein referred to as BCY19599);
Ac-(SEQ ID NO: 204)-[K(PYA)] (herein referred to as BCY19600);
Ac-(SEQ ID NO: 204)-[K(PYA)]-triazolyl-PEG₁₀-amido-PIB (herein referred to as BCY20014);
Ac-(SEQ ID NO: 205)-[K(PYA)] (herein referred to as BCY19638);
Ac-(SEQ ID NO: 206)-[K(PYA)] (herein referred to as BCY19639);
Ac-(SEQ ID NO: 207)-[K(PYA)] (herein referred to as BCY19640);
Ac-(SEQ ID NO: 208)-[K(PYA)] (herein referred to as BCY19641);
Ac-(SEQ ID NO: 209)-[K(PYA)] (herein referred to as BCY19654);
Ac-(SEQ ID NO: 210)-[K(PYA)] (herein referred to as BCY19655);
Ac-(SEQ ID NO: 211)-[K(PYA)] (herein referred to as BCY19658);
Ac-(SEQ ID NO: 212)-[K(PYA)] (herein referred to as BCY19827);
Ac-(SEQ ID NO: 213)-[K(PYA)] (herein referred to as BCY19990); and
Ac-(SEQ ID NO: 214)-[K(PYA)] (herein referred to as BCY20268);
   wherein PYA represents pentynoic acid and PIB represents 4 (4-iodophenyl) butyrate.

In a still yet further embodiment, said loop sequences comprise three reactive groups separated by two loop sequences one of which consists of 8 amino acids and the other of which consists of 2 amino acids, the molecular scaffold is TCMT, the bicyclic peptide ligand additionally comprises N- and/or C-terminal additions and comprises an amino acid sequence which is selected from:
A-(SEQ ID NO: 36)-A (herein referred to as BCY15342);
A-(SEQ ID NO: 37)-A (herein referred to as BCY15343);
Ac-A-(SEQ ID NO: 37)-A (herein referred to as BCY16322);
Ac-(SEQ ID NO: 37) (herein referred to as BCY16926);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321); and
Ac-(SEQ ID NO: 88) (herein referred to as BCY16591).

In a still yet further embodiment, said loop sequences comprise three reactive groups separated by two loop sequences one of which consists of 8 amino acids and the other of which consists of 2 amino acids, the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 37)-A-[Sar₆]-[KFl] (herein referred to as BCY15318);
Ac-(SEQ ID NO: 37)-[Sar₆]-[KFl] (herein referred to as BCY16323);
A-(SEQ ID NO: 38)-A-[Sar₆]-[KFl] (herein referred to as BCY15319); and
Ac-(SEQ ID NO: 88)-A-[Sar₆]-[KFl] (herein referred to as BCY16679).

In a still yet further embodiment, said loop sequences comprise three reactive groups separated by two loop sequences one of which consists of 8 amino acids and the other of which consists of 2 amino acids, the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 37)-A-[Sar₆]-[KFl] (herein referred to as BCY15318);
A-(SEQ ID NO: 38)-A-[Sar₆]-[KFl] (herein referred to as BCY15319); and
Ac-(SEQ ID NO: 88)-A-[Sar₆]-[KFl] (herein referred to as BCY16679).

In a still yet further embodiment, said loop sequences comprise three reactive groups separated by two loop sequences one of which consists of 8 amino acids and the other of which consists of 2 amino acids, the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 37)-A-[Sar₆]-[KFl] (herein referred to as BCY15318); and
A-(SEQ ID NO: 38)-A-[Sar₆]-[KFl] (herein referred to as BCY15319).

In an alternative embodiment, said loop sequences comprise three reactive groups separated by two loop sequences one of which consists of 8 amino acids and the other of which consists of 3 amino acids.

In a further embodiment, said loop sequences comprise three reactive groups separated by two loop sequences one of which consists of 8 amino acids and the other of which consists of 3 amino acids and the bicyclic peptide ligand comprises an amino acid sequence which is selected from:

(SEQ ID NO: 45)
CᵢSNTWHWTDCᵢᵢLAECᵢᵢᵢ;
and
(SEQ ID NO: 47)
CᵢNLWNGDPWCᵢᵢLLRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 8 amino acids and the other of which consists of 3 amino acids, the molecular scaffold is TATA, the bicyclic peptide ligand additionally comprises N- and/or C-terminal additions and comprises an amino acid sequence which is selected from:
A-(SEQ ID NO: 45)-A (herein referred to as BCY15351); and
A-(SEQ ID NO: 47)-A (herein referred to as BCY15353).

In a still yet further embodiment, said loop sequences comprise three reactive groups separated by two loop sequences one of which consists of 8 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 45)-A-[Sar₆]-[KFl] (herein referred to as BCY15322); and
A-(SEQ ID NO: 47)-A-[Sar₆]-[KFl] (herein referred to as BCY15323).

In an alternative embodiment, said loop sequences comprise three reactive groups separated by two loop sequences one of which consists of 8 amino acids and the other of which consists of 4 amino acids.

In a further embodiment, said loop sequences comprise three reactive groups separated by two loop sequences one of which consists of 8 amino acids and the other of which consists of 4 amino acids and the bicyclic peptide ligand comprises an amino acid sequence which is selected from:

(SEQ ID NO: 42)
CᵢHQLMDIWDCᵢᵢLRPDCᵢᵢᵢ;
and
(SEQ ID NO: 43)
CᵢLTAREKIQCᵢᵢLQRRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 8 amino acids and the other of which consists of 4 amino acids, the molecular scaffold is TCMT, the bicyclic peptide ligand additionally comprises N- and/or C-terminal additions and comprises an amino acid sequence which is selected from:
A-(SEQ ID NO: 42)-A (herein referred to as BCY15348); and
A-(SEQ ID NO: 43)-A (herein referred to as BCY15349).

In a still yet further embodiment, said loop sequences comprise three reactive groups separated by two loop sequences one of which consists of 8 amino acids and the other of which consists of 2 amino acids, the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 42)-A-[Sar₆]-[KFl] (herein referred to as BCY15320); and
A-(SEQ ID NO: 43)-A-[Sar₆]-[KFl] (herein referred to as BCY15321).

In one particular embodiment, the peptide ligand of the invention is selected from BCY15324, BCY16679, BCY15299, BCY15437, BCY15310, BCY16298 and BCY16287. These peptide ligands are believed to bind to 7 different epitopes of the S1 spike protein (referred to internally as Epitopes 1, 2, 3, 4, 5, 9 and 10, respectively). More crucially, the peptide ligands of this embodiment provide the significant advantage of being able to bind to a variety of differing mutant/variants of COVID-19 S1 spike protein as evidenced in the data presented herein as Example 3.

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within peptides of the invention, cysteine residues (Cᵢ, Cᵢᵢ and Cᵢᵢᵢ) are omitted from the numbering as they are invariant, therefore, the numbering of amino acid residues within peptides of the invention is referred to as below:

(SEQ ID NO: 1)
Cᵢ-H₁-H₂-A₃-Cᵢᵢ-P₄-I₅-L₆-T₇-G₈-W₉-Cᵢᵢᵢ.

For the purpose of this description, all bicyclic peptides are assumed to be cyclised with TATA, TATB or TCMT and yielding a tri-substituted structure. Cyclisation with TATA, TATB or TCMT occurs on the first, second and third reactive groups (i.e. Cᵢ, Cᵢᵢ, Cᵢᵢᵢ).

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A-.

### Inversed Peptide Sequences

In light of the disclosure in Nair et al (2003) J Immunol 170 (3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

### Half-Life Extending Moieties

In one embodiment, the peptide ligand may additionally comprise a half-life extending moiety in order to extend and improve the half-life of the resultant peptide ligand. One such example of a half-life extending moiety is a polyethylene glycol (PEG) moiety, such as triazolyl-PEG₁₀-amido-PIB (wherein PIB represents 4 (4-iodophenyl) butyrate). BCY20014 is an example of a bicyclic peptide ligand of the invention described herein which contains this half-life extending moiety.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. Certain ligands demonstrate cross-reactivity across Lipid II from different bacterial species and hence are able to treat infections caused by multiple species of bacteria. Other ligands may be highly specific for the Lipid II of certain bacterial species which may be advantageous for treating an infection without collateral damage to the beneficial flora of the patient;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and

### Selectivity.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (+)-DL-lactic), lactobionic, maleic, malic, (−)-L-malic, malonic, (+)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH⁴⁺) and substituted ammonium ions (e.g., NH₃R⁺, NH₂R²⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency. Alternatively, non-natural amino acids may be used having constrained amino acid side chains, such that proteolytic hydrolysis of the nearby peptide bond is conformationally and sterically impeded. In particular, these concern proline analogues, bulky sidechains, Co-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102 (2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio) isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio) isotopes, and peptide ligands of the invention, wherein certain functional groups are covalently replaced with relevant (radio) isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene or iodoacetamide. Other scaffold reactive groups that are used to selectively couple compounds to cysteines in proteins are maleimides, αβ unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene.

In one embodiment, the molecular scaffold is selected from 1,1′,1″-(1,3,5-triazinane-1,3,5-triyl)triprop-2-en-1-one (also known as triacryloylhexahydro-s-triazine; TATA), 1,3,5-tris(bromoacetyl) hexahydro-1,3,5-triazine (TATB) and 2,4,6-tris(chloromethyl)-s-triazine (TCMT).

In a further embodiment, the molecular scaffold is 1,1′,1″-(1,3,5-triazinane-1,3,5-triyl)triprop-2-en-1-one (also known as triacryloylhexahydro-s-triazine (TATA):

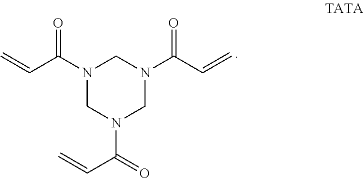 [SMILES: C=CC(=O)N1CN(C(=O)C=C)CN(C(=O)C=C)C1]

Thus, following cyclisation with the bicyclic peptides of the invention on the Cᵢ, Cᵢᵢ, and Cᵢᵢᵢ cysteine residues, the molecular scaffold forms a tri-substituted 1,1′,1″-(1,3,5-triazinane-1,3,5-triyl)tripropan-1-one derivative of TATA having the following structure:

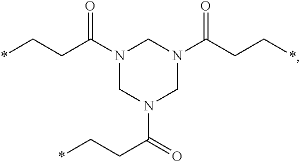

wherein * denotes the point of attachment of the three cysteine residues.

In an alternative embodiment, the molecular scaffold is 1,3,5-tris(bromoacetyl) hexahydro-1, 3,5-triazine (TATB):

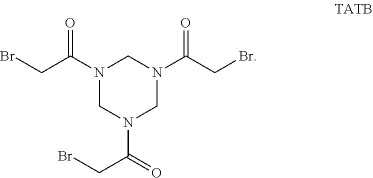 [SMILES: O=C(CBr)N1CN(C(=O)CBr)CN(C(=O)CBr)C1]

Thus, following cyclisation with the bicyclic peptides of the invention on the Cᵢ, Cᵢᵢ, and Cᵢᵢᵢ cysteine residues, the molecular scaffold forms a tri-substituted 1,3,5-tris(bromoacetyl) hexahydro-1,3,5-triazine derivative of TATB having the following structure:

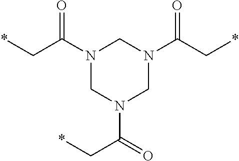 [SMILES: *CC(=O)N1CN(C(=O)C*)CN(C(=O)C*)C1]

wherein * denotes the point of attachment of the three cysteine residues.

In an alternative embodiment, the molecular scaffold is 2,4,6-tris(chloromomethyl)-s-triazine (TCMT):

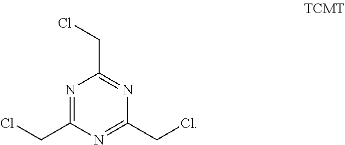 [SMILES: ClCc1nc(CCl)nc(CCl)n1]

Thus, following cyclisation with the bicyclic peptides of the invention on the Cᵢ, Cᵢᵢ, and Cᵢᵢᵢ cysteine residues, the molecular scaffold forms a tri-substituted 2,4,6-tris(chloromomethyl)-s-triazine derivative of TCMT having the following structure:

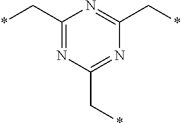 [SMILES: *Cc1nc(C*)nc(C*)n1]

wherein * denotes the point of attachment of the three cysteine residues.

Details of chemistryrelated to TCMT and the use of the corresponding bromide and its use in the formation of cyclic peptides are described in van de Langemheen et al (2016) ChemBioChem 10.1002/cbic.201600612 (https://onlinelibrary.wiley.com/doi/abs/10.1002/cbic.2016006.12).

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a [Dap(Me)] group, a lysine side chain, or an N-terminal amine group or any other suitable reactive group. Details may be found in WO 2009/098450. In one embodiment, the reactive groups are all cysteine residues.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment of the invention, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, an polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5 (7), 502-7.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides selected as set out herein, wherein the manufacture comprises optional further steps as explained below. In one embodiment, these steps are carried out on the end product polypeptide made by chemical synthesis.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio) conjugation techniques may be used to introduce an activated or activatable N- or C-terminus. Alternatively, additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91 (26): 12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA, TATB or TCMT) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the invention can be used alone or in combination with another agent or agents.

The compounds of the invention can also be used in combination with biological therapies such as nucleic acid based therapies, antibodies, bacteriophage or phage lysins.

The route of administration of pharmaceutical compositions according to the invention may be any of those commonly known to those of ordinary skill in the art. For therapy, the peptide ligands of the invention can be administered to any patient in accordance with standard techniques. Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Preferably, the pharmaceutical compositions according to the invention will be administered parenterally. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 10 μg to 250 mg of selected peptide ligand per kilogram of body weight, with doses of between 100 μg to 25 mg/kg/dose being more commonly used.

A composition containing a peptide ligand according to the present invention may be utilised in therapeutic settings to treat a microbial infection or to provide prophylaxis to a subject at risk of infection e.g. undergoing surgery, chemotherapy, artificial ventilation or other condition or planned intervention. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as severe acute respiratory syndrome coronavirus 2 (SARS-COV-2) binding agents.

Polypeptide ligands selected according to the method of the present invention may be employed in in vivo therapeutic applications, in vitro and in vivo diagnostic applications, in vitro assay and reagent applications, and the like. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand as defined herein, for use in suppressing or treating a disease or disorder mediated by infection of SARS-CoV-2 or for providing prophylaxis to a subject at risk of infection of SARS-COV-2.

According to a further aspect of the invention, there is provided a method of suppressing or treating a disease or disorder mediated by infection of SARS-COV-2 or for providing prophylaxis to a subject at risk of infection of SARS-COV-2, which comprises administering to a patient in need thereof the peptide ligand as defined herein.

References herein to “disease or disorder mediated by infection of SARS-COV-2” include: respiratory disorders, such as a respiratory disorder mediated by an inflammatory response within the lung, in particular COVID-19.

References herein to the term “suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available.

### Screening Methods

It will be appreciated that the bicyclic peptide ligands of the invention also find utility as agents for screening for other SARS-COV-2 binding agents.

For example, screening for a SARS-COV-2 binding agent may typically involve incubating a bicyclic peptide ligand of the invention with SARS-COV-2 in the presence and absence of a test compound and assessing a difference in the degree of binding, such that a difference in binding will result from competition of the test compound with the bicyclic peptide ligand of the invention for binding to SARS-COV-2.

Thus, according to a further aspect of the invention, there is provided a method of screening for a compound which binds to SARS-COV-2 wherein said method comprises the following steps:
(a) incubating a peptide ligand as defined herein with SARS-COV-2;
(b) measuring the binding activity of said peptide ligand;
(c) incubating said peptide ligand from step (a) with a test compound and SARS-CoV-2;
(d) measuring the binding activity of said peptide ligand; and
(e) comparing the binding activity in steps (b) and (d), such that a difference in binding activity of said peptide ligand is indicative of the test compound binding to SARS-COV-2.

In one embodiment, the peptide ligand comprises a reporter moiety for ease of detecting binding. In a further embodiment, the reporter moiety comprises fluorescein (Fl). In a yet further embodiment, the peptide ligand comprises any of the peptide ligands described herein which comprise a fluorescein (Fl) moiety.

### Diagnostic Methods

It will be appreciated that the bicyclic peptide ligands of the invention also find utility as agents for diagnosing infection of SARS-COV-2.

For example, diagnosis of SARS-COV-2 infection may typically involve incubating a bicyclic peptide ligand of the invention with SARS-COV-2 in the presence and absence of a test compound and assessing a difference in the degree of binding, such that a difference in binding will result from competition of the test compound with the bicyclic peptide ligand of the invention for binding to SARS-COV-2.

Thus, according to a further aspect of the invention, there is provided a method of diagnosing SARS-COV-2 infection wherein said method comprises the following steps:
a) obtaining a biological sample from an individual;
(b) incubating a peptide ligand as defined herein with the biological sample obtained in step (a); and
(c) detecting binding of said peptide ligand to SARS-COV-2, such that a detection of measurable binding activity is indicative of a diagnosis of SARS-COV-2 infection.

In one embodiment, the peptide ligand comprises a reporter moiety for ease of detecting binding. In a further embodiment, the reporter moiety comprises fluorescein (Fl). In a yet further embodiment, the peptide ligand comprises any of the peptide ligands described herein which comprise a fluorescein (Fl) moiety.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology.

Alternatively, peptides were purified using HPLC and following isolation they were modified with the required molecular scaffold (namely, TATA, TATB or TCMT). For this, linear peptide was diluted with 50:50 MeCN:H₂O up to ˜35 mL, ˜500 μL of 100 mM scaffold in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA, TATB or TCMT.

Following lyophilisation, the modified peptide was purified as above, while replacing the Luna C8 with a Gemini C18 column (Phenomenex), and changing the acid to 0.1% trifluoroacetic acid. Pure fractions containing the correct scaffold-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio) pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Biological Data

### Example 1: Affinity Determination by Fluorescence Polarization (FP) Direct Binding

Bicycles labelled with fluorescein (tracers) were screened in a fluorescence polarisation direct binding assay to determine affinity (K_{d}) forwild-type (WT) S1 domain (ACROBiosystems, S1N-C82E8) of the SARS-COV-2 Spike Protein. Tracers were added at 1 nM final to a titration of individual SARS-COV-2 Spike Protein variants in assay buffer (PBS+0.01% Tween20, pH7.4) to a maximum of 2.54 μM. Fluorescence was measured at 485/520/520 on a BMG PHERAstar FSX plate reader. Where appropriate, SARS-COV-2 Spike protein variants alone parallel and perpendicular intensities were subtracted before mP was calculated. Subsequently, mP data was fit to non-linear regression analysis in Dotmatics to generate a K_{d} value. Where no significant assay window was generated, data was reported to show no binding at maximum concentration of protein. Where a K_{d} was generated above the top concentration of protein tested, the result was flagged as K_{d} greater than maximum concentration of protein tested-results associated with this flag may be displayed as K_{d}>x μM.

Selected bicyclic peptides of the invention were tested in the above mentioned direct binding assay and the results are shown in Table 1:

TABLE 1
Direct Binding Assay Results for Selected
Bicyclic Peptides of the Invention

| Bicyclic Peptide | K_{d} (μM) |
|---|---|
| BCY15318 | 0.7168 |
| BCY 16312 | 0.7591 |
| BCY 16313 | 0.3964 |
| BCY 16314 | 0.518 |
| BCY 16315 | 0.7775 |
| BCY 16316 | >1 |
| BCY 16318 | 0.6018 |
| BCY 16319 | 0.7258 |
| BCY 16320 | 0.3605 |
| BCY 16321 | 0.9906 |
| BCY 16322 | 0.7221 |
| BCY 16323 | 0.7125 |
| BCY 16679 | 0.339 |
| BCY 15310 | 0.1986 |
| BCY 16538 | 0.2287 |
| BCY 16534 | 0.3812 |
| BCY 16535 | >1 |
| BCY 16536 | 0.424 |
| BCY 16537 | >0.963 |
| BCY 16545 | 0.641 |
| BCY 16544 | 0.921 |
| BCY 16543 | >1 |
| BCY 16542 | 0.511 |
| BCY 16541 | >1 |
| BCY 16540 | >0.883 |
| BCY 15522 | 0.37 |

### Example 2: Surface Plasmon Resonance (SPR) Assay

Selected bicyclic peptides of the invention were tested in an SPR assay in accordance with standard procedures known to those skilled in the art and the results are shown in Table 2:

TABLE 2
SPR Assay Results for Selected Bicyclic Peptides of the Invention

| Bicyclic Peptide | pK_{D} |
|---|---|
| BCY 15576 | 6.88 |
| BCY 16903 | 7.92 |
| BCY 16905 | 7.98 |
| BCY 16906 | 7.68 |
| BCY 16911 | 8 |
| BCY 16913 | 7.77 |
| BCY 16915 | 7.81 |
| BCY 16917 | 7.96 |
| BCY 16918 | 7.94 |
| BCY 16921 | 7.85 |
| BCY 16912 | 7.72 |
| BCY 16914 | 7.78 |
| BCY 16916 | 7.75 |
| BCY 16919 | 7.68 |
| BCY 16920 | 7.69 |
| BCY 16902 | 7.62 |
| BCY 16904 | 7.24 |
| BCY 16907 | 7.46 |
| BCY 16908 | 7.47 |
| BCY 16909 | 7.58 |
| BCY 15251 | 7.65 |

### Example 3: Affinity Determination by Fluorescence Polarization (FP) Direct Binding Using Mutated Variants of Spike Protein

This experiment was conducted in the same manner as described in Example 1 to assess binding of selected bicyclic peptides of the invention against a selection of mutated variants of the isolated S1, S1-NTD and S1-RBD domains of spike protein. The results are shown in Table 3 below.

TABLE 3
Direct Binding Assay Results for Selected
Bicyclic Peptides of the Invention

| Peptide | Epi- | | Tested COVID-19 S1 |
|---|---|---|---|
| Number | tope | K_{d} (μM) | Spike Protein Mutant |
| BCY15324 | 1 | No binding at max conc | N354D, D364Y |
| | | 0.588 | G476S |
| | | 0.751 | V483A |
| | | 1.49 | E484K |
| | | 0.629 | A475V |
| | | No binding at max conc | L452R |
| | | No binding at max conc | F490L |
| | | No binding at max conc | K417N, E484K, N501Y |
| | | 0.529 | N439K |
| | | 0.366 | Y453F |
| | | 0.715 | N440K |
| | | 1.26 | N501Y |
| BCY16679 | 2 | 0.294 | N354D, D364Y |
| | | 0.194 | W436R |
| | | 0.263 | R408I |
| | | 0.305 | V367F |
| | | 0.38 | D614G |
| | | 0.253 | G476S |
| | | 0.294 | V483A |
| | | No binding at max conc | E484K |
| | | 0.134 | A475V |
| | | No binding at max conc | L452R |
| | | No binding at max conc | F490L |
| | | No binding at max conc | K417N, E484K, N501Y |
| | | 0.19 | N439K |
| | | 0.52 | Y453F |
| | | 0.13 | N440K |
| | | 0.402 | N501Y |
| BCY15299 | 3 | No binding at max conc | N354D, D364Y |
| | | 0.944 | G476S |
| | | 0.988 | V483A |
| | | 0.475 | E484K |
| | | 0.71 | A475V |
| | | 0.548 | L452R |
| | | 0.441 | F490L |
| | | 0.676 | K417N, E484K, N501Y |
| | | 0.531 | N439K |
| | | 0.579 | Y453F |
| | | 0.539 | N440K |
| | | 0.935 | N501Y |
| BCY15437 | 4 | 0.167 | Y144del |
| | | 0.024 | A222V |
| | | 0.024 | N234Q |
| | | 0.015 | A262S |
| | | 0.017 | P272L |
| | | 0.043 | HV69-70del |
| | | 0.019 | L18F, D80A, D215G, |
| | | | R246I |
| BCY15310 | 5 | 0.285 | D614G |
| | | 0.169 | HV69-70del, N501Y, |
| | | | D614G |
| | | 0.305 | P681H |
| | | 0.265 | A570D |
| BCY16298 | 9 | K_{d} greater than | N354D, D364Y |
| | | max conc tested | |
| | | 1.31 | G476S |
| | | K_{d} greater than | V483A |
| | | max conc tested | |
| | | 0.865 | E484K |
| | | K_{d} greater than | A475V |
| | | max conc tested | |
| | | 1.15 | L452R |
| | | 1.22 | F490L |
| | | K_{d} greater than | K417N, E484K, N501Y |
| | | max conc tested | |
| | | K_{d} greater than | N439K |
| | | max conc tested | |
| | | 1.02 | Y453F |
| | | 1.17 | N440K |
| | | K_{d} greater than | N501Y |
| | | max conc tested | |
| BCY16287 | 10 | 1.12 | N354D, D364Y |
| | | 0.058 | G476S |
| | | 0.069 | V483A |
| | | 0.055 | E484K |
| | | 0.054 | A475V |
| | | 0.053 | L452R |
| | | 0.049 | F490L |

The effectiveness of the tested peptides from Table 3 against the many S1 spike protein mutants may be summarised in Table 4:

TABLE 4
Summary of Binding Status of Tested Bicyclic Peptides to S1 Spike Protein Variants

| | Peptide | | | | | | |
|---|---|---|---|---|---|---|---|
| Variant | BCY15324 | BCY16679 | BCY15299 | BCY15437 | BCY15310 | BCY16298 | BCY16287 |
| N354D, D364Y | NB | ✓ | NB | nt | nt | ~ | ✓ |
| G476S | ✓ | ✓ | ✓ | nt | nt | ✓ | ✓ |
| V483A | ✓ | ✓ | ✓ | nt | nt | ~ | ✓ |
| E484K | ✓ | NB | ✓ | nt | nt | ✓ | ✓ |
| A475V | ✓ | ✓ | ✓ | nt | nt | ~ | ✓ |
| L452R | NB | NB | ✓ | nt | nt | ✓ | ✓ |
| F490L | NB | NB | ✓ | nt | nt | ✓ | ✓ |
| K417N, E484K, N501Y | NB | NB | ✓ | nt | nt | ~ | nt |
| N439K | ✓ | ✓ | ✓ | nt | nt | ~ | nt |
| Y453F | ✓ | ✓ | ✓ | nt | nt | ✓ | nt |
| N440K | ✓ | ✓ | ✓ | nt | nt | ✓ | nt |
| N501Y | ✓ | ✓ | ✓ | nt | nt | ~ | nt |
| W436R | nt | ✓ | nt | nt | nt | nt | nt |
| R408I | nt | ✓ | nt | nt | nt | nt | nt |
| V367F | nt | ✓ | nt | nt | nt | nt | nt |
| D614G | nt | ✓ | nt | nt | ✓ | nt | nt |
| Y144del | nt | nt | nt | ✓ | nt | nt | nt |
| A222V | nt | nt | nt | ✓ | nt | nt | nt |
| N234Q | nt | nt | nt | ✓ | nt | nt | nt |
| A262S | nt | nt | nt | ✓ | nt | nt | nt |
| P272L | nt | nt | nt | ✓ | nt | nt | nt |
| HV69-70del | nt | nt | nt | ✓ | nt | nt | nt |
| L18F, D80A, D215G, R246I | nt | nt | nt | ✓ | nt | nt | nt |
| HV69-70del, N501Y, D614G | nt | nt | nt | nt | ✓ | nt | nt |
| P681H | nt | nt | nt | nt | ✓ | nt | nt |
| A570D | nt | nt | nt | nt | ✓ | nt | nt |

Legend
nt = not tested
✓ = measurable binding
~ = K_{d} greater than maximum concentration tested - binding observed
NB = no binding at maximum concentration

The results of Example 3 demonstrate that the tested bicyclic peptides demonstrated excellent binding activity to the mutated variants of the S1spike protein with only several instances of no binding observed within the limits of the assay.

### Example 4: Affinity Determination by Surface Plasmon Resonance (SPR) Against Multiple Spike Protein Constructs

SPR assays were performed on a Biacore T200 (Cytiva) with Series S Streptavidin (SA) sensor chips (Cytiva) in assay buffer (pH7.4, 10 mM HEPES, 150 mM NaCl, 3 mM EDTA, 0.05% (v/v) Surfactant P20, 2% DMSO) or alternatively nickel chloride activated Sesies S Sensor Chip NTA (Cytiva) in assay buffer (PBS-P⁺ (Cytiva), 1% DMSO). Spike protein constructs (ACROBiosystems) protein was captured to generate 3000-4000 RU. Peptide binding was performed at 25° C., using a 30 μl/min flow rate with appropriate association and dissociation periods. Bicycles were assayed at concentrations between no greater than 10000 nM in either a multiple or single cycle kinetic format. All data were double-referenced against blank injections and reference surface (treated with assay buffer) using standard processing procedures. Regeneration of streptavidin surface was performed using 1 mM HCl (30 s at 30 μL/min) followed by a stabilization period of 30 s. Each injection was followed by an additional wash with 50% DMSO, to reduce Bicycle carry-over. Kinetic values (kₐ, k_{d} and K_{O}) were derived from the sensorgrams by applying 1:1 Binding or Steady-State analysis, using the Biacore™ T200 Evaluation Software (version 3.1).

Selected bicyclic peptides of the invention were tested in the above mentioned SPR assay and the results are shown in Table 5:

TABLE 5
SPR Assay Results for Selected Bicyclic Peptides of the Invention

| | Geomean | Spike Protein |
|---|---|---|
| BCY Number | Average KD (nM) | Construct |
| BCY15251 | 69.1 | Biotinylated 2019-nCoV |
| BCY15343 | 2240 | (COVID-19) S1 protein |
| BCY15444 | 2180 | (16-685), His, Avitag |
| BCY15445 | 482 | |
| BCY15446 | 1060 | |
| BCY15576 | 89.9 | |
| BCY16207 | 8300 | |
| BCY16591 | 545 | |
| BCY16886 | 154 | |
| BCY16887 | 95.3 | |
| BCY16889 | 44.8 | |
| BCY16895 | 43.7 | |
| BCY16902 | 24.0 | |
| BCY16903 | 11.1 | |
| BCY16904 | 58.2 | |
| BCY16905 | 10.0 | |
| BCY16906 | 17.8 | |
| BCY16907 | 34.4 | |
| BCY16908 | 33.9 | |
| BCY16909 | 26.3 | |
| BCY16910 | 22.6 | |
| BCY16911 | 12.6 | |
| BCY16912 | 19.0 | |
| BCY16913 | 15.5 | |
| BCY16914 | 16.5 | |
| BCY16915 | 17.9 | |
| BCY16916 | 17.9 | |
| BCY16917 | 10.7 | |
| BCY16918 | 11.3 | |
| BCY16919 | 20.8 | |
| BCY16920 | 20.3 | |
| BCY16921 | 14.0 | |
| BCY16926 | 2470 | |
| BCY16927 | 18.8 | |
| BCY16930 | 59.1 | |
| BCY16933 | 25.3 | |
| BCY16940 | 207 | |
| BCY16941 | 83.9 | |
| BCY16942 | 92.0 | |
| BCY16946 | 78.1 | |
| BCY16948 | 9.79 | |
| BCY17279 | 55.2 | |
| BCY17281 | 105 | |
| BCY17282 | 99.5 | |
| BCY17283 | 225 | |
| BCY17287 | 1390 | |
| BCY17289 | 44.1 | |
| BCY17294 | 76.3 | |
| BCY17301 | 77.0 | |
| BCY17302 | 187 | |
| BCY17303 | 109 | |
| BCY17304 | 122 | |
| BCY17305 | 67.3 | |
| BCY17306 | 37.9 | |
| BCY17307 | 73.3 | |
| BCY17308 | 127 | |
| BCY17309 | 95.3 | |
| BCY17310 | 86.5 | |
| BCY17311 | 33.6 | |
| BCY17313 | 1350 | |
| BCY17359 | 2250 | |
| BCY17540 | 1810 | |
| BCY17541 | 245 | |
| BCY17542 | 574 | |
| BCY17543 | 178 | |
| BCY17544 | 350 | |
| BCY17547 | 933 | |
| BCY17548 | 1610 | |
| BCY18024 | 800 | |
| BCY18025 | 898 | |
| BCY18026 | 1160 | |
| BCY18027 | 1660 | |
| BCY18040 | 2220 | |
| BCY18086 | 1710 | |
| BCY18087 | 1220 | |
| BCY18088 | 793 | |
| BCY18109 | 945 | |
| BCY18110 | 829 | |
| BCY18111 | 914 | |
| BCY18115 | 224 | |
| BCY18211 | 813 | |
| BCY18212 | 1060 | |
| BCY18340 | 20.4 | |
| BCY18341 | 66.7 | |
| BCY18342 | 31.3 | |
| BCY18343 | 71.5 | |
| BCY18344 | 12.7 | |
| BCY18345 | 34.7 | |
| BCY18346 | 20.6 | |
| BCY18347 | 50.3 | |
| BCY18351 | 717 | |
| BCY18524 | 102 | |
| BCY18527 | 1720 | |
| BCY18529 | 1240 | |
| BCY18654 | 1280 | |
| BCY18661 | 1600 | |
| BCY18662 | 2610 | |
| BCY18698 | 8900 | |
| BCY18707 | 2440 | |
| BCY19305 | 3500 | |
| BCY19309 | 220 | |
| BCY19378 | 70.9 | |
| BCY19599 | 44.5 | |
| BCY19600 | 0.588 | |
| BCY19638 | 11.9 | |
| BCY19639 | 34.4 | |
| BCY19640 | 350 | |
| BCY19641 | 199 | |
| BCY19654 | 384 | |
| BCY19655 | 98.5 | |
| BCY19658 | 61.0 | |
| BCY19827 | 0.485 | |
| BCY19990 | 66.1 | |
| BCY20014 | 1.38 | |
| BCY20268 | 0.462 | |
| BCY16591 | 458 | Biotinylated SARS- |
| BCY19309 | 186 | CoV-2 (COVID-19) S |
| BCY19533 | 6510 | protein RBD, |
| BCY19534 | 5230 | His, Avitag ™ |
| BCY19535 | 565 | |
| BCY19536 | 7570 | |
| BCY19537 | 2650 | |
| BCY19538 | 2100 | |
| BCY19539 | 2080 | |
| BCY19541 | 2170 | |
| BCY19542 | 1950 | |
| BCY19543 | 2940 | |
| BCY19544 | 6650 | |
| BCY19545 | 1570 | |
| BCY19546 | 3520 | |
| BCY19547 | 9160 | |
| BCY19548 | 2990 | |
| BCY19599 | 61.6 | |
| BCY19600 | 0.834 | |
| BCY19638 | 29.5 | |
| BCY19639 | 72.0 | |
| BCY19654 | 115 | |
| BCY19655 | 169 | |
| BCY19658 | 96.6 | |
| BCY20014 | 0.379 | |
| BCY19600 | 457 | Biotinylated SARS- |
| BCY20014 | 192 | CoV-2 S protein RBD |
| | | (K417T, E484K, N501Y), |
| | | His, Avitag ™ |
| BCY20014 | 311 | Biotinylated SARS- |
| | | CoV-2 S protein RBD |
| | | (K417N, E484K, N501Y), |
| | | His, Avitag ™ |
| BCY19600 | 30100 | Biotinylated SARS- |
| BCY20014 | 7620 | CoV-2 Spike RBD |
| | | (L452R, E484Q), |
| | | His, Avitag ™ |

### Example 5: Pseudovirus Neutralisation Assay

Replication deficient SARS-COV-2 pseudotyped HIV-1 virions were prepared similarly as described in Mallery et al (2021) Sci Adv 7 (11). Briefly, virions were produced in HEK 293T cells by transfection with 1 μg of the plasmid encoding SARS COV-2 Spike protein (pCAGGS-SpikeΔc19), 1 μg pCRV GagPol and 1.5 μg GFP-encoding plasmid (CSGW). Viral supernatants were filtered through a 0.45 μm syringe filter at 48 h and 72 h post-transfection and pelleted for 2 h at 28,000×g. Pelleted virions were drained and then resuspended in DMEM (Gibco).

HEK 293T-hACE2-TMPRSS2 cells were prepared as described in Papa et al (2021) PLOS Pathog 17 (1): p. e1009246. Cells were plated into 96-well plates at a density of 2×103 cells per well in Free style 293T expression media and allowed to attach overnight. 18 μl pseudovirus-containing supernatant was mixed with 2 μl dilutions of bicyclic peptide and incubated for 40 min at RT. 10 μl of this mixture was added to cells. 72 h later, cell entry was detected through the expression of GFP by visualisation on an Incucyte S3 live cell imaging system (Sartorius). The percent of cell entry was quantified as GFP positive areas of cells over the total area covered by cells. Entry inhibition by the Bicycle was calculated as percent virus infection relative to virus only control.

Selected bicyclic peptides of the invention were tested in the above mentioned Pseudovirus Neutralisation Assay and the results are shown in Table 6:

TABLE 6
Pseudovirus Neutralisation Assay Results for
Selected Bicyclic Peptides of the Invention

| BCY Number | Geomean IC₅₀ (nM) | Pseudovirus Construct |
|---|---|---|
| BCY19600 | 183 | Wild-Type |

### Example 6: SARS-COV-2 Cytopathic Effect (CPE)

A549_ACE_TMPRSS2 cells were seeded in 96-well plates and cultured overnight. The following day, 4-fold serial dilutions of the bicyclic peptides were prepared in medium and 60 μl of the diluted compounds starting from a maximum concentration of 30, 15, 10, 3, 1, or 0.1 μM were added to the plates with cells. After 3 h pre-incubation, cells were infected with SARS-COV-2 GLA-1 at MOI 0.04 PFU/cell. One dose of 522 PFU of the virus in 60 μl per well was added to the wells containing compounds. Plates were incubated for 72 h at 37° C., fixed and stained when the cytopathic effect (CPE) was visible. Plates were scanned in a plate reader to quantitate the levels of CPE.

Selected bicyclic peptides of the invention were tested in the above mentioned Cytopathic Effect Assay and the results are shown in Table 7:

TABLE 7
Cytopathic Effect Assay Results for Selected
Bicyclic Peptides of the Invention

| BCY Number | IC₅₀ (nM) |
|---|---|
| BCY16591 | 21400 |
| BCY18024 | 29000 |
| BCY20014 | 168 |

1. A peptide ligand specific for severe acute respiratory syndrome coronavirus 2 (SARS-COV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The peptide ligand according to claim 1, wherein said peptide ligand is specific for the spike protein (S protein) of SARS-COV-2.

3. The peptide ligand according to claim 1, wherein said peptide ligand is specific for the S1 of S2 domain of the spike protein (S protein), such as the S1 domain of the spike protein (S1 protein).

4. The peptide ligand according to claim 1, wherein said loop sequences comprise 2, 3, 4, 5, 6, 7 or 8 amino acids.

5. The peptide ligand according to claim 1, wherein said loop sequences comprise three reactive groups separated by two loop sequences one of which consists of 3 amino acids and the other of which consists of 6 amino acids, such as:
wherein the bicyclic peptide ligand comprises an amino acid sequence which is selected from:
(SEQ ID NO: 1)
CᵢHHACᵢᵢPILTGWCᵢᵢᵢ;
(SEQ ID NO: 6)
CᵢPHACᵢᵢPSLWGWCᵢᵢᵢ;
(SEQ ID NO: 7)
CᵢLHACᵢᵢPRLTHWCᵢᵢᵢ;
(SEQ ID NO: 8)
CᵢLHACᵢᵢQYLWGYCᵢᵢᵢ;
(SEQ ID NO: 9)
CᵢSHACᵢᵢPRLFGWCᵢᵢᵢ;
(SEQ ID NO: 10)
CᵢQHACᵢᵢPYLWDYCᵢᵢᵢ;
(SEQ ID NO: 58)
CᵢPFACᵢᵢHKLYGWCᵢᵢᵢ;
(SEQ ID NO: 59)
CᵢMKACᵢᵢPYLYGWCᵢᵢᵢ;
(SEQ ID NO: 60)
CᵢRHACᵢᵢTHLYGHCᵢᵢᵢ;
(SEQ ID NO: 61)
CᵢPYACᵢᵢTRLYGWCᵢᵢᵢ;
(SEQ ID NO: 62)
CᵢSHACᵢᵢPRLTGWCᵢᵢᵢ;
(SEQ ID NO: 63)
CᵢLHSCᵢᵢPRLSGWCᵢᵢᵢ;
(SEQ ID NO: 64)
CᵢRHSCᵢᵢPILTGWCᵢᵢᵢ;
(SEQ ID NO: 65)
CᵢGHSCᵢᵢPVLWGWCᵢᵢᵢ;
(SEQ ID NO: 66)
CᵢPHSCᵢᵢPKLFGWCᵢᵢᵢ;
(SEQ ID NO: 67)
CᵢTHSCᵢᵢPYLFGWCᵢᵢᵢ;
(SEQ ID NO: 118)
CᵢDWTCᵢᵢYLTMMPCᵢᵢᵢ;
(SEQ ID NO: 119)
CᵢDWTCᵢᵢYLRPLPCᵢᵢᵢ;
(SEQ ID NO: 120)
CᵢDWTCᵢᵢYMSMKPCᵢᵢᵢ;
(SEQ ID NO: 121)
CᵢDWTCᵢᵢYFRPLPCᵢᵢᵢ;
and
(SEQ ID NO: 122)
CᵢDWTCᵢᵢYISPMFDCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15230);
A-(SEQ ID NO: 6)-A (herein referred to as BCY15235);
A-(SEQ ID NO: 7)-A (herein referred to as BCY15236);
A-(SEQ ID NO: 8)-A (herein referred to as BCY15237);
A-(SEQ ID NO: 9)-A (herein referred to as BCY15238);
A-(SEQ ID NO: 10)-A (herein referred to as BCY15239);
A-(SEQ ID NO: 58)-A (herein referred to as BCY15364);
A-(SEQ ID NO: 59)-A (herein referred to as BCY15365);
A-(SEQ ID NO: 60)-A (herein referred to as BCY15366);
A-(SEQ ID NO: 61)-A (herein referred to as BCY15367);
A-(SEQ ID NO: 62)-A (herein referred to as BCY15368);
A-(SEQ ID NO: 63)-A (herein referred to as BCY15369);
A-(SEQ ID NO: 64)-A (herein referred to as BCY15370);
A-(SEQ ID NO: 65)-A (herein referred to as BCY15371);
A-(SEQ ID NO: 66)-A (herein referred to as BCY15372); and
A-(SEQ ID NO: 67)-A (herein referred to as BCY15373);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 6)-A-[Sar₆]-[KFl] (herein referred to as BCY15303); and
A-(SEQ ID NO: 63)-A-[Sar₆]-[KFl] (herein referred to as BCY15329);
or wherein the molecular scaffold is TATB and the bicyclic peptide ligand additionally comprises N- and/or C-terminal additions and comprises an amino acid sequence which is selected from:
A-(SEQ ID NO: 118)-A (herein referred to as BCY15444);
A-(SEQ ID NO: 119)-A (herein referred to as BCY16927);
A-(SEQ ID NO: 120)-A (herein referred to as BCY16930);
A-(SEQ ID NO: 121)-A (herein referred to as BCY16933); and
A-(SEQ ID NO: 122)-A (herein referred to as BCY16940).

6. The peptide ligand according to claim 1, wherein said loop sequences comprise three reactive groups separated by two loop sequences one of which consists of 3 amino acids and the other of which consists of 7 amino acids, such as:
wherein the bicyclic peptide ligand comprises an amino acid sequence which is selected from:
(SEQ ID NO: 123)
CᵢDWTCᵢᵢYLNIYHECᵢᵢᵢ;
(SEQ ID NO: 124)
CᵢDWTCᵢᵢYMDYLSNCᵢᵢᵢ;
(SEQ ID NO: 125)
CᵢDWTCᵢᵢYLRIHEACᵢᵢᵢ;
(SEQ ID NO: 126)
CᵢDWTCᵢᵢYMRINDACᵢᵢᵢ;
and
(SEQ ID NO: 127)
CᵢDWTCᵢᵢYINIYNTCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATB and the bicyclic peptide ligand additionally comprises N- and/or C-terminal additions and comprises an amino acid sequence which is selected from:
A-(SEQ ID NO: 123)-A (herein referred to as BCY15445);
A-(SEQ ID NO: 124)-A (herein referred to as BCY16941);
A-(SEQ ID NO: 125)-A (herein referred to as BCY16942); and
A-(SEQ ID NO: 126)-A (herein referred to as BCY16946);
or wherein the molecular scaffold is TCMT and the bicyclic peptide ligand additionally comprises N- and/or C-terminal additions and comprises an amino acid sequence which is:
A-(SEQ ID NO: 127)-A (herein referred to as BCY16948).

7. The peptide ligand according to claim 1, wherein said loop sequences comprise three reactive groups separated by two loop sequences one of which consists of 4 amino acids and the other of which consists of 6 amino acids, such as:
wherein the bicyclic peptide ligand comprises an amino acid sequence which is selected from:
(SEQ ID NO: 29)
CᵢᵢLTNDCᵢᵢHSDIRYCᵢᵢᵢ;
and
(SEQ ID NO: 30)
CᵢITNDCᵢᵢHTSLIFCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TCMT and the bicyclic peptide ligand additionally comprises N- and/or C-terminal additions and comprises an amino acid sequence which is selected from:
A-(SEQ ID NO: 29)-A (herein referred to as BCY15335); and
A-(SEQ ID NO: 30)-A (herein referred to as BCY15336);
or wherein the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 30)-A-[Sar₆]-[KFl] (herein referred to as BCY15314).

8. The peptide ligand according to claim 1, wherein said loop sequences comprise three reactive groups separated by two loop sequences one of which consists of 4 amino acids and the other of which consists of 8 amino acids, such as:
wherein the bicyclic peptide ligand comprises an amino acid sequence which is selected from:
(SEQ ID NO: 3)
CᵢVDANCᵢᵢKIKILQRMCᵢᵢᵢ;
(SEQ ID NO: 4)
CᵢTSSVCᵢᵢKIKELQRKCᵢᵢᵢ;
(SEQ ID NO: 5)
CᵢRSLLCᵢᵢEYLQRTDSCᵢᵢᵢ;
(SEQ ID NO: 14)
CᵢLTKSCᵢᵢKIKMLQRVCᵢᵢᵢ;
(SEQ ID NO: 15)
CᵢMQPSCᵢᵢRVLQLQRVCᵢᵢᵢ;
(SEQ ID NO: 16)
CᵢALPSCᵢᵢRILHLQHRCᵢᵢᵢ;
(SEQ ID NO: 17)
CᵢHDAHCᵢᵢKILELQHRCᵢᵢᵢ;
(SEQ ID NO: 18)
CᵢTSSHCᵢᵢRVLEEQRLCᵢᵢᵢ;
(SEQ ID NO: 19)
CᵢPRDRCᵢᵢPTAWLYGLCᵢᵢᵢ;
(SEQ ID NO: 20)
CᵢAEAGCᵢᵢRVKQLQQICᵢᵢᵢ;
(SEQ ID NO: 21)
CᵢTPSPCᵢᵢRVKELQRACᵢᵢᵢ;
(SEQ ID NO: 26)
CᵢSTANCᵢᵢRILELQQLCᵢᵢᵢ;
(SEQ ID NO: 44)
CᵢVGRLCᵢᵢSTATDIRKCᵢᵢᵢ;
(SEQ ID NO: 48; herein referred to as
BCY16983 when complexed with TATB)
CᵢRQSQCᵢᵢDWWAIRSFCᵢᵢᵢ;
(SEQ ID NO: 49)
CᵢTDATCᵢᵢSIKRLQRLCᵢᵢᵢ;
(SEQ ID NO: 50)
CᵢSPVSCᵢᵢPSGFKFGLCᵢᵢᵢ;
(SEQ ID NO: 68)
CᵢDSPWCᵢᵢRIRSLQRQCᵢᵢᵢ;
(SEQ ID NO: 69)
CᵢSVGACᵢᵢRVKLLQRVCᵢᵢᵢ;
(SEQ ID NO: 70)
CᵢMFVPCᵢᵢAVREILGLCᵢᵢᵢ;
(SEQ ID NO: 128)
CᵢSDLMCᵢᵢWYLQRTDSCᵢᵢᵢ;
(SEQ ID NO: 129)
CᵢNSYMCᵢᵢWYLQRTDSCᵢᵢᵢ;
(SEQ ID NO: 130)
CᵢTSYLCᵢᵢWYLQRTDSCᵢᵢᵢ;
and
(SEQ ID NO: 131)
CᵢRSLMCᵢᵢWYLNQTDSCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATB and the bicyclic peptide ligand additionally comprises N- and/or C-terminal additions and comprises an amino acid sequence which is selected from:
A-(SEQ ID NO: 3)-A (herein referred to as BCY15334);
A-(SEQ ID NO: 15)-A (herein referred to as BCY15244);
A-(SEQ ID NO: 16)-A (herein referred to as BCY15245);
A-(SEQ ID NO: 17)-A (herein referred to as BCY15246);
A-(SEQ ID NO: 18)-A (herein referred to as BCY15247);
A-(SEQ ID NO: 19)-A (herein referred to as BCY15248);
A-(SEQ ID NO: 20)-A (herein referred to as BCY15249);
A-(SEQ ID NO: 21)-A (herein referred to as BCY15250);
A-(SEQ ID NO: 26)-A (herein referred to as BCY15255);
A-(SEQ ID NO: 48)-A (herein referred to as BCY15354);
A-(SEQ ID NO: 48)-A (herein referred to as BCY16534);
A-(SEQ ID NO: 48)-AK (herein referred to as BCY16896);
A-(SEQ ID NO: 49)-A (herein referred to as BCY15355); and
A-(SEQ ID NO: 50)-A (herein referred to as BCY15356);
or wherein the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15301);
A-(SEQ ID NO: 15)-A-[Sar₆]-[KFl] (herein referred to as BCY15307);
A-(SEQ ID NO: 17)-A-[Sar₆]-[KFl] (herein referred to as BCY15308);
A-(SEQ ID NO: 19)-A-[Sar₆]-[KFl] (herein referred to as BCY15309);
A-(SEQ ID NO: 48)-A-[Sar₆]-[KFl] (herein referred to as BCY15324);
A-(SEQ ID NO: 49)-A-[Sar₆]-[KFl] (herein referred to as BCY15325); and
A-(SEQ ID NO: 50)-A-[Sar₆]-[KFl] (herein referred to as BCY15326);
or wherein the molecular scaffold is TATA, the bicyclic peptide ligand additionally comprises N- and/or C-terminal additions and comprises an amino acid sequence which is selected from:
A-(SEQ ID NO: 3)-A (herein referred to as BCY15232);
A-(SEQ ID NO: 4)-A (herein referred to as BCY15233);
A-(SEQ ID NO: 5)-A (herein referred to as BCY15234);
A-(SEQ ID NO: 14)-A (herein referred to as BCY15243);
A-(SEQ ID NO: 44)-A (herein referred to as BCY15350);
A-(SEQ ID NO: 68)-A (herein referred to as BCY15374);
A-(SEQ ID NO: 69)-A (herein referred to as BCY15375);
A-(SEQ ID NO: 70)-A (herein referred to as BCY15376);
A-(SEQ ID NO: 128)-A (herein referred to as BCY16886);
A-(SEQ ID NO: 129)-A (herein referred to as BCY16887);
A-(SEQ ID NO: 130)-A (herein referred to as BCY16889); and
A-(SEQ ID NO: 131)-A (herein referred to as BCY16895);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15300);
A-(SEQ ID NO: 5)-A-[Sar₆]-[KFl] (herein referred to as BCY15302); and
A-(SEQ ID NO: 70)-A-[Sar₆]-[KFl] (herein referred to as BCY15330).

9. The peptide ligand according to claim 1, wherein said loop sequences comprise three reactive groups separated by two loop sequences one of which consists of 5 amino acids and the other of which consists of 3 amino acids, such as:
wherein the bicyclic peptide ligand comprises an amino acid sequence which is:
(SEQ ID NO: 115)
CᵢFDDWTCᵢᵢYIQMCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 115)-A-[Sar₆]-[KFl] (herein referred to as BCY15437).

10. The peptide ligand according to claim 1, wherein said loop sequences comprise three reactive groups separated by two loop sequences one of which consists of 6 amino acids and the other of which consists of 3 amino acids, such as:
wherein the bicyclic peptide ligand comprises an amino acid sequence which is selected from:
(SEQ ID NO: 71)
CᵢTLMDPWCᵢᵢLLKCᵢᵢᵢ;
(SEQ ID NO: 72)
CᵢKIHDWTCᵢᵢLLRCᵢᵢᵢ;
and
(SEQ ID NO: 79; herein referred to as
BCY18707 when complexed with TATB)
CᵢIPLDWTCᵢᵢMIACᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 71)-A (herein referred to as BCY15377); and
A-(SEQ ID NO: 72)-A (herein referred to as BCY15378);
or wherein the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 71)-A-[Sar₆]-[KFl] (herein referred to as BCY15331);
or wherein the molecular scaffold is TATB and the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446);
A-(SEQ ID NO: 79)-AK (herein referred to as BCY16994); and
Ac-(SEQ ID NO: 79)-K (herein referred to as BCY18654).

11. The peptide ligand according to claim 1, wherein said loop sequences comprise three reactive groups separated by two loop sequences one of which consists of 6 amino acids and the other of which consists of 4 amino acids, such as:
wherein the bicyclic peptide ligand comprises an amino acid sequence which is selected from:
(SEQ ID NO: 11)
CᵢEYQGPHCᵢᵢYRLYCᵢᵢᵢ;
(SEQ ID NO: 132)
CᵢEYNGPYCᵢᵢYRLYCᵢᵢᵢ;
and
(SEQ ID NO: 133)
CᵢEYVGPMCᵢᵢYRLYCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N- and/or C-terminal additions and comprises an amino acid sequence which is selected from:
A-(SEQ ID NO: 11)-A (herein referred to as BCY15240);
A-(SEQ ID NO: 132)-A (herein referred to as BCY17547); and
A-(SEQ ID NO: 133)-A (herein referred to as BCY17548);
or wherein the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 11)-A-[Sar₆]-[KFl] (herein referred to as BCY15304).

12. The peptide ligand according to claim 1, wherein said loop sequences comprise three reactive groups separated by two loop sequences one of which consists of 7 amino acids and the other of which consists of 2 amino acids, such as:
wherein the bicyclic peptide ligand comprises an amino acid sequence which is selected from:
(SEQ ID NO: 2)
CᵢEDHDWVYCᵢᵢSTCᵢᵢᵢ;
(SEQ ID NO: 23)
CᵢAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
(SEQ ID NO: 75)
CᵢPNISWIYCᵢᵢSTCᵢᵢᵢ;
(SEQ ID NO: 117)
CᵢDVCᵢᵢGLNAFNRCᵢᵢᵢ;
(SEQ ID NO: 134)
CᵢLDETWIYCᵢᵢSTCᵢᵢᵢ;
(SEQ ID NO: 135)
CᵢPDETWVYCᵢᵢSTCᵢᵢᵢ;
(SEQ ID NO: 136)
CᵢESNDWVYCᵢᵢSTCᵢᵢᵢ;
(SEQ ID NO: 137)
CᵢEDNDWVYCᵢᵢSTCᵢᵢᵢ;
and
(SEQ ID NO: 138)
CᵢPDVSWIYCᵢᵢSTCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N- and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380);
A-(SEQ ID NO: 75)-A (herein referred to as BCY15381);
A-(SEQ ID NO: 134)-A (herein referred to as BCY17540);
A-(SEQ ID NO: 135)-A (herein referred to as BCY17541);
A-(SEQ ID NO: 136)-A (herein referred to as BCY17542);
A-(SEQ ID NO: 137)-A (herein referred to as BCY17543); and
A-(SEQ ID NO: 138)-A (herein referred to as BCY17544);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 2)-A-[Sar₆]-[KFl] (herein referred to as BCY15299); and
A-(SEQ ID NO: 74)-A-[Sar₆]-[KFl] (herein referred to as BCY15332);
or wherein the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 117)-A-[Sar₆]-[KFl] (herein referred to as BCY16287);
or wherein the molecular scaffold is TATB, the bicyclic peptide ligand additionally comprises N- and/or C-terminal additions and comprises an amino acid sequence which is selected from:
A-(SEQ ID NO: 23)-A (herein referred to as BCY15252);
A-(SEQ ID NO: 25)-A (herein referred to as BCY15254);
A-(SEQ ID NO: 28)-A (herein referred to as BCY15257);
A-(SEQ ID NO: 51)-A (herein referred to as BCY15357);
A-(SEQ ID NO: 52)-A (herein referred to as BCY15358);
A-(SEQ ID NO: 53)-A (herein referred to as BCY15359);
A-(SEQ ID NO: 54)-A (herein referred to as BCY15360); and
A-(SEQ ID NO: 55)-A (herein referred to as BCY15361);
or wherein the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 23)-A-[Sar₆]-[KFl] (herein referred to as BCY15311);
A-(SEQ ID NO: 25)-A-[Sar₆]-[KFl] (herein referred to as BCY15312); and
A-(SEQ ID NO: 53)-A-[Sar₆]-[KFl] (herein referred to as BCY15327).

13. The peptide ligand according to claim 1, wherein said loop sequences comprise three reactive groups separated by two loop sequences one of which consists of 7 amino acids and the other of which consists of 3 amino acids, such as:
wherein the bicyclic peptide ligand comprises an amino acid sequence which is selected from:
(SEQ ID NO: 22; herein referred to as
BCY16534 when complexed with TATB)
CᵢASPDNPVCᵢᵢRFYCᵢᵢᵢ;
(SEQ ID NO: 24; herein referred to as
BCY16540 when complexed with TATB)
CᵢYNHANPVCᵢᵢRYYCᵢᵢᵢ;
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CᵢHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢCASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
(SEQ ID NO: 114)
CᵢYNHANPVCᵢᵢ[Arg(Me)]YYCᵢᵢᵢ;
(SEQ ID NO: 116)
CᵢMNPFFYDCᵢᵢERTCᵢᵢᵢ;
(SEQ ID NO: 139)
Cᵢ[Aib]SPDNPVCᵢᵢRFYCᵢᵢᵢ;
(SEQ ID NO: 140)
CᵢAS[HyP]DNPVCᵢᵢRFYCᵢᵢᵢ;
(SEQ ID NO: 141)
CᵢAS[Aib]DNPVCᵢᵢRFYCᵢᵢᵢ;
(SEQ ID NO: 142)
CᵢAS[Pip]DNPVCᵢᵢRFYCᵢᵢᵢ;
(SEQ ID NO: 143)
CᵢᵢASPDN[Pip]VCᵢᵢRFYCᵢᵢᵢ;
(SEQ ID NO: 144)
CᵢASPDN[44DFP]VCᵢᵢRFYCᵢᵢᵢ;
(SEQ ID NO: 145)
CᵢASPDN[4FlPro]VCᵢᵢRFYCᵢᵢᵢ;
(SEQ ID NO: 146)
CᵢASPDNPVCᵢᵢR[1Nal]YCᵢᵢᵢ;
(SEQ ID NO: 147)
CᵢASPDNPVCᵢᵢR[2Nal]YCᵢᵢᵢ;
(SEQ ID NO: 148)
CᵢASPDNPVCᵢᵢR[2MePhe]YCᵢᵢᵢ;
(SEQ ID NO: 149)
CᵢASPDNPVCᵢᵢR[3MePhe]YCᵢᵢᵢ;
(SEQ ID NO: 150)
CᵢASPDNPVCᵢᵢR[4MePhe]YCᵢᵢᵢ;
(SEQ ID NO: 151)
CᵢASPDNPVCᵢᵢR[2ClPhe]YCᵢᵢᵢ;
(SEQ ID NO: 152)
CᵢASPDNPVCᵢᵢR[3ClPhe]YCᵢᵢᵢ;
(SEQ ID NO: 153)
CᵢASPDNPVCᵢᵢR[4ClPhe]YCᵢᵢᵢ;
(SEQ ID NO: 154)
CᵢASPDNPVCᵢᵢR[2FPhe]YCᵢᵢᵢ;
(SEQ ID NO: 155)
CᵢASPDNPVCᵢᵢR[3FPhe]YCᵢᵢᵢ;
(SEQ ID NO: 156)
CᵢASPDNPVCᵢᵢR[4FPhe]YCᵢᵢᵢ;
(SEQ ID NO: 157)
CᵢASPDNPVCᵢᵢR[26DiMeTyr]YCᵢᵢᵢ;
(SEQ ID NO: 158)
C[Aib]SPDN[44DFP]VCᵢᵢR[4FPhe]YCᵢᵢᵢ;
(SEQ ID NO: 159)
Cᵢ[Aib]SPDN[44DFP]VCᵢᵢ[Arg(Me)][4FPhe]YCᵢᵢᵢ;
(SEQ ID NO: 160)
Cᵢ[Aib]SPDNPVCᵢᵢR[4FPhe]YCᵢᵢᵢ;
(SEQ ID NO: 161)
Cᵢ[Aib]SPDNPVCᵢᵢ[Arg(Me)][4FPhe]YCᵢᵢᵢ;
(SEQ ID NO: 162)
Cᵢ[Aib]NPDN[44DFP]VCᵢᵢR[4FPhe]YCᵢᵢᵢ;
(SEQ ID NO: 163)
Cᵢ[Aib]NPDN[44DFP]VCᵢᵢ[Arg(Me)][4FPhe]YCᵢᵢᵢ;
(SEQ ID NO: 164)
Cᵢ[Aib]NPDNPVCᵢᵢR[4FPhe]YCᵢᵢᵢ;
and
(SEQ ID NO: 165)
Cᵢ[Aib]NPDNPVCᵢᵢ[Arg(Me)][4FPhe]YCᵢᵢᵢ;
wherein Cᵢ, Cᵢᵢ and Cᵢᵢᵢ represent first, second and third cysteine residues, respectively, Aib represents aminoisobutyric acid, Agb represents 2-amino-4-guanidinobutyric acid, Arg(Me) represents d-N methyl arginine, 2ClPhe represents 2-chloro-phenylalanine, 3ClPhe represents 3-chloro-phenylalanine, 4ClPhe represents 4-chloro-phenylalanine, 44DFP represents 4,4-difluoroproline, 26DiMeTyr represents 2,6-dimethyl-tyrosine, 2FPhe represents 2-fluoro-phenylalanine, 3FPhe represents 3-fluoro-phenylalanine, 4FPhe represents 4-fluoro-phenylalanine, 4FlPro represents 4-fluoro-proline, HArg represents homoarginine, HyP represents hydroxyproline, 2MePhe represents 2-methyl-phenylalanine, 3MePhe represents 3-methyl-phenylalanine, 4MePhe represents 4-methyl-phenylalanine, 1Nal represents 1-naphthylalanine, 2Nal represents 2-naphthylalanine, Pip represents pipecolic acid, or a pharmaceutically acceptable salt thereof, in particular:
wherein the molecular scaffold is TATB, the bicyclic peptide ligand additionally comprises N- and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543);
A-(SEQ ID NO: 114)-A (herein referred to as BCY16542);
A-(SEQ ID NO: 116)-A (herein referred to as BCY16207);
Ac-(SEQ ID NO: 116) (herein referred to as BCY18698);
Ac-(SEQ ID NO: 139) (herein referred to as BCY17279);
Ac-(SEQ ID NO: 140) (herein referred to as BCY17281);
Ac-(SEQ ID NO: 141) (herein referred to as BCY17282);
Ac-(SEQ ID NO: 142) (herein referred to as BCY17283);
Ac-(SEQ ID NO: 143) (herein referred to as BCY17287);
Ac-(SEQ ID NO: 144) (herein referred to as BCY17289);
Ac-(SEQ ID NO: 145) (herein referred to as BCY17294);
Ac-(SEQ ID NO: 146) (herein referred to as BCY17301);
Ac-(SEQ ID NO: 147) (herein referred to as BCY17302);
Ac-(SEQ ID NO: 148) (herein referred to as BCY17303);
Ac-(SEQ ID NO: 149) (herein referred to as BCY17304);
Ac-(SEQ ID NO: 150) (herein referred to as BCY17305);
Ac-(SEQ ID NO: 151) (herein referred to as BCY17306);
Ac-(SEQ ID NO: 152) (herein referred to as BCY17307);
Ac-(SEQ ID NO: 153) (herein referred to as BCY17308);
Ac-(SEQ ID NO: 154) (herein referred to as BCY17309);
Ac-(SEQ ID NO: 155) (herein referred to as BCY17310);
Ac-(SEQ ID NO: 156) (herein referred to as BCY17311);
Ac-(SEQ ID NO: 157) (herein referred to as BCY17313);
Ac-(SEQ ID NO: 158) (herein referred to as BCY18340);
Ac-(SEQ ID NO: 159) (herein referred to as BCY18341);
Ac-(SEQ ID NO: 160) (herein referred to as BCY18342);
Ac-(SEQ ID NO: 161) (herein referred to as BCY18343);
Ac-(SEQ ID NO: 162) (herein referred to as BCY18344);
Ac-(SEQ ID NO: 163) (herein referred to as BCY18345);
Ac-(SEQ ID NO: 164) (herein referred to as BCY18346); and
Ac-(SEQ ID NO: 165) (herein referred to as BCY18347);
or wherein the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 22)-A-[Sar₆]-[KFl] (herein referred to as BCY15310);
A-(SEQ ID NO: 27)-A-[Sar₆]-[KFl] (herein referred to as BCY15313);
A-(SEQ ID NO: 56)-A-[Sar₆]-[KFl] (herein referred to as BCY15328); and
A-(SEQ ID NO: 116)-A-[Sar₆]-[KFl] (herein referred to as BCY16298);
or wherein the molecular scaffold is TCMT, the bicyclic peptide ligand additionally comprises N- and/or C-terminal additions and comprises an amino acid sequence which is:
A-(SEQ ID NO: 31)-A (herein referred to as BCY15315);
or wherein the molecular scaffold is TCMT, the bicyclic peptide additionally comprises N- and/or C-terminal additions and a labelling moiety, such as fluorescein (Fl), and comprises an amino acid sequence which is:
A-(SEQ ID NO: 31)-A-[Sar₆]-[KFl] (herein referred to as BCY15313);
or wherein the molecular scaffold is TATA, the bicyclic peptide ligand additionally comprises N- and/or C-terminal additions and comprises an amino acid sequence which is:
A-(SEQ ID NO: 76)-A (herein referred to as BCY15382);
or wherein the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 76)-A-[Sar₆]-[KFl] (herein referred to as BCY15333).

14. The peptide ligand according to claim 1, wherein said loop sequences comprise three reactive groups separated by two loop sequences one of which consists of 7 amino acids and the other of which consists of 5 amino acids, such as:
wherein the bicyclic peptide ligand comprises an amino acid sequence which is selected from:
(SEQ ID NO: 32)
CᵢTTSEKVKCᵢᵢLQRHPCᵢᵢᵢ;
(SEQ ID NO: 33)
CᵢQPDMRIKCᵢᵢLQRVACᵢᵢᵢ;
(SEQ ID NO: 34)
CᵢSSNNRIKCᵢᵢLQRVTCᵢᵢᵢ;
(SEQ ID NO: 35)
CᵢKEKTTIGCᵢᵢLMAGICᵢᵢᵢ;
and
(SEQ ID NO: 166)
CᵢNRPTSVYCᵢᵢLQRGICᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TCMT, the bicyclic peptide ligand additionally comprises N- and/or C-terminal additions and comprises an amino acid sequence which is selected from:
A-(SEQ ID NO: 32)-A (herein referred to as BCY15338);
A-(SEQ ID NO: 33)-A (herein referred to as BCY15339);
A-(SEQ ID NO: 34)-A (herein referred to as BCY15340);
A-(SEQ ID NO: 35)-A (herein referred to as BCY15341); and
A-(SEQ ID NO: 166)-A (herein referred to as BCY17359);
or wherein the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 32)-A-[Sar₆]-[KFl] (herein referred to as BCY15316); and
A-(SEQ ID NO: 33)-A-[Sar₆]-[KFl] (herein referred to as BCY15317).

15. The peptide ligand according to claim 1, wherein said loop sequences comprise three reactive groups separated by two loop sequences one of which consists of 8 amino acids and the other of which consists of 2 amino acids, such as:
wherein the bicyclic peptide ligand comprises an amino acid sequence which is selected from:
(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as
BCY16312 when complexed with TCMT)
CᵢPYVAGRGTCᵢᵢLLCᵢᵢᵢ;
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
(SEQ ID NO: 88)
CᵢPYVAG[Agb][dA]TCᵢᵢL[tBuAla]Cᵢᵢᵢ;
(SEQ ID NO: 167; herein referred to as
BCY18111 when complexed with TCMT)
CᵢPYVAG[HArg][dA]TCᵢᵢL[tBuAla]Cᵢᵢᵢ;
(SEQ ID NO: 168)
CᵢPYVPG[HArg][dA]TCᵢᵢL[tBuAla]Cᵢᵢᵢ;
(SEQ ID NO: 169)
Cᵢ[K(PYA)]YVAG[HArg][dA]TCᵢᵢL[tBuAla]Cᵢᵢᵢ;
(SEQ ID NO: 170)
Cᵢ[HyP]YVAG[HArg][dA]TCᵢᵢL[tBuAla]Cᵢᵢᵢ;
(SEQ ID NO: 171)
CᵢPYVAGT[dA]TCᵢᵢL[tBuAla]Cᵢᵢᵢ;
(SEQ ID NO: 172)
CᵢPYVAG[Agb][dA]TCᵢᵢLLCᵢᵢᵢ;
(SEQ ID NO: 173)
CᵢPYVAG[Agb]GTCᵢᵢL[tBuAla]Cᵢᵢᵢ;
(SEQ ID NO: 174)
CᵢPYV[HyP]G[HArg][dA]TCᵢᵢL[tBuAla]Cᵢᵢᵢ;
(SEQ ID NO: 175)
CᵢPY[K(PYA)]AG[HArg][dA]TCᵢᵢL[tBuAla]Cᵢᵢᵢ;
(SEQ ID NO: 176)
Cᵢ[Oic]YVAG[HArg][dA]TCᵢᵢL[tBuAla]Cᵢᵢᵢ;
(SEQ ID NO: 177)
CᵢPY[B-Melle]AG[HArg][dA]TCᵢᵢL[tBuAla]Cᵢᵢᵢ;
(SEQ ID NO: 178)
CᵢPY[tBuGly]AG[HArg][dA]TCᵢᵢL[tBuAla]Cᵢᵢᵢ;
(SEQ ID NO: 179)
CᵢPYPAG[HArg][dA]TCᵢᵢL[tBuAla]Cᵢᵢᵢ;
(SEQ ID NO: 180)
CᵢP[44BPA]VAG[HArg][dA]TCᵢᵢL[tBuAla]Cᵢᵢᵢ;
(SEQ ID NO: 181)
CᵢP[2FPhe]VAG[HArg][dA]TCᵢᵢL[tBuAla]Cᵢᵢᵢ;
(SEQ ID NO: 182)
CᵢPY[Cba]AG[HArg][dA]TCᵢᵢL[tBuAla]Cᵢᵢᵢ;
(SEQ ID NO: 183)
CᵢP[3FPhe]VAG[HArg][dA]TCᵢᵢL[tBuAla]Cᵢᵢᵢ;
(SEQ ID NO: 184)
Cᵢ[55DMP]YVAG[HArg][dA]TCᵢᵢL[tBuAla]Cᵢᵢᵢ;
(SEQ ID NO: 185)
CᵢPYVAGQ[dA]TCᵢᵢL[tBuAla]Cᵢᵢᵢ;
(SEQ ID NO: 186)
CᵢPYVAG[HArg][dS]TCᵢᵢL[tBuAla]Cᵢᵢᵢ;
(SEQ ID NO: 187)
CᵢP[4tBuPhe]VAG[HArg][dA]TCᵢᵢL[tBuAla]Cᵢᵢᵢ;
(SEQ ID NO: 188)
CᵢPYREGTGTCᵢᵢLLCᵢᵢᵢ;
(SEQ ID NO: 189)
CᵢPYAPGNGTCᵢᵢLLCᵢᵢᵢ;
(SEQ ID NO: 190)
CᵢPHPPGRGTCᵢᵢLLCᵢᵢᵢ;
(SEQ ID NO: 191)
CᵢPYNAGTGTCᵢᵢLLCᵢᵢᵢ;
(SEQ ID NO: 192)
CᵢPYSPGQGTCᵢᵢLLCᵢᵢᵢ;
(SEQ ID NO: 193)
CᵢPYQPGSGTCᵢᵢLLCᵢᵢᵢ;
(SEQ ID NO: 194)
CᵢPFPPGMGTCᵢᵢLLCᵢᵢᵢ;
(SEQ ID NO: 195)
CᵢPHQPGFGTCᵢᵢLLCᵢᵢᵢ;
(SEQ ID NO: 196)
CᵢPYSPGSGTCᵢᵢLLCᵢᵢᵢ;
(SEQ ID NO: 197)
CᵢPYLAGTGTCᵢᵢLLCᵢᵢᵢ;
(SEQ ID NO: 198)
CᵢPWEAGKGTCᵢᵢLLCᵢᵢᵢ;
(SEQ ID NO: 199)
CᵢPYAPGMGTCᵢᵢLLCᵢᵢᵢ;
(SEQ ID NO: 200)
CᵢPHMPGSGTCᵢᵢLLCᵢᵢᵢ;
(SEQ ID NO: 201)
CᵢPYNKGEGTCᵢᵢLLCᵢᵢᵢ;
(SEQ ID NO: 202)
CᵢPFKPGVGTCᵢᵢLLCᵢᵢᵢ;
(SEQ ID NO: 203)
CᵢP[4tBuPhe]VAG[Orn][dA]TCᵢᵢL[tBuAla]Cᵢᵢᵢ;
(SEQ ID NO: 204)
Cᵢ[Oic][4tBuPhe]VAG[HArg][dA]TCᵢᵢL[tBuAla]Cᵢᵢᵢ;
(SEQ ID NO: 205)
CᵢP[4tBuPhe]VAG[Dab][dA]TCᵢᵢL[tBuAla]Cᵢᵢᵢ;
(SEQ ID NO: 206)
CᵢP[4tBuPhe]VAG[Dap][dA]TCᵢᵢL[tBuAla]Cᵢᵢᵢ;
(SEQ ID NO: 207)
CᵢP[4CF3Phe]VAG[HArg][dA]TCᵢᵢL[tBuAla]Cᵢᵢᵢ;
(SEQ ID NO: 208)
CᵢP[DMAPhe]VAG[HArg][dA]TCᵢᵢL[tBuAla]Cᵢᵢᵢ;
(SEQ ID NO: 209)
CᵢP[4tBuPhe]VAG[HArg][dDab]TCᵢᵢL[tBuAla]Cᵢᵢᵢ;
(SEQ ID NO: 210)
CᵢP[4tBuPhe]VAG[HSer][dA]TCᵢᵢL[tBuAla]Cᵢᵢᵢ;
(SEQ ID NO: 211)
CᵢP[4tBuPhe]VAG[Cit][dA]TCᵢᵢL[tBuAla]Cᵢᵢᵢ;
(SEQ ID NO: 212)
C[Oic][4tBuPhe]VAG[Orn][dA]TCᵢᵢL[tBuAla]Cᵢᵢᵢ;
(SEQ ID NO: 213)
CᵢP[4tBuPhe]VAG[HArg][dDap]TCᵢᵢL[tBuAla]Cᵢᵢᵢ;
and
(SEQ ID NO: 214)
Cᵢ[Oic][4tBuPhe]VAG[Cit][dA]TCᵢᵢL[tBuAla]Cᵢᵢᵢ;
wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, B-Melle represents β-methyl-isoleucine, 44BPA represents 4,4-biphenylalanine, Cba represents β-cyclobutylalanine, 4CF3Phe represents 4-trifluoromethyl-phenylalanine, Cit represents citrulline, Dab represents diaminobutanoic acid, Dap represents diaminopropionic acid, DMAPhe represents 4-Dimethylamino-phenylalanine, 55DMP represents 5,5-Dimethyl-L-proline, 2FPhe represents 2-fluoro-phenylalanine, 3FPhe represents 3-fluoro-phenylalanine, HArg represents homoarginine, HSer represents homoserine, HyP represents hydroxyproline, Oic represents octahydroindolecarboxylic acid, Orn represents ornithine, PYA represents pentynoic acid, tBuAla represents t-butyl-alanine, tBuGly represents t-butyl-glycine, 4tBuPhe represents 4-t-butyl-phenylalanine, or a pharmaceutically acceptable salt thereof, in particular:
wherein the molecular scaffold is TATA, the bicyclic peptide ligand additionally comprises N- and/or C-terminal additions and comprises an amino acid sequence which is selected from:
A-(SEQ ID NO: 12)-A (herein referred to as BCY15241);
A-(SEQ ID NO: 13)-A (herein referred to as BCY15242);
A-(SEQ ID NO: 77)-A (herein referred to as BCY15383); and
A-(SEQ ID NO: 78)-A (herein referred to as BCY15384);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 12)-A-[Sar₆]-[KFl] (herein referred to as BCY15305); and
A-(SEQ ID NO: 13)-A-[Sar₆]-[KFl] (herein referred to as BCY15306);
or wherein the molecular scaffold is TCMT, the bicyclic peptide ligand additionally comprises N- and/or C-terminal additions and comprises an amino acid sequence which is selected from:
A-(SEQ ID NO: 36)-A (herein referred to as BCY15342);
Ac-A-(SEQ ID NO: 37)-A (herein referred to as BCY16322);
Ac-(SEQ ID NO: 37) (herein referred to as BCY16926);
A-(SEQ ID NO: 37)-A (herein referred to as BCY15343);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
Ac-(SEQ ID NO: 80) (herein referred to as BCY18086);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321);
Ac-(SEQ ID NO: 88) (herein referred to as BCY16591);
Ac-(SEQ ID NO: 167) (herein referred to as BCY18024);
Ac-(SEQ ID NO: 168)-[K(PYA)] (herein referred to as BCY18025);
Ac-(SEQ ID NO: 169) (herein referred to as BCY18026);
Ac-(SEQ ID NO: 170)-[K(PYA)] (herein referred to as BCY18027);
Ac-(SEQ ID NO: 171)-[K(PYA)] (herein referred to as BCY18040);
Ac-(SEQ ID NO: 172) (herein referred to as BCY18087);
Ac-(SEQ ID NO: 173) (herein referred to as BCY18088);
Ac-(SEQ ID NO: 174)-[K(PYA)] (herein referred to as BCY18109);
Ac-(SEQ ID NO: 175) (herein referred to as BCY18110);
Ac-(SEQ ID NO: 176)-[K(PYA)] (herein referred to as BCY18115);
Ac-(SEQ ID NO: 177)-[K(PYA)] (herein referred to as BCY18211);
Ac-(SEQ ID NO: 178)-[K(PYA)] (herein referred to as BCY18212);
Ac-(SEQ ID NO: 179)-[K(PYA)] (herein referred to as BCY18351);
Ac-(SEQ ID NO: 180)-[K(PYA)] (herein referred to as BCY18524);
Ac-(SEQ ID NO: 181)-[K(PYA)] (herein referred to as BCY18527);
Ac-(SEQ ID NO: 182)-[K(PYA)] (herein referred to as BCY18529);
Ac-(SEQ ID NO: 183)-[K(PYA)] (herein referred to as BCY18661);
Ac-(SEQ ID NO: 184)-[K(PYA)] (herein referred to as BCY18662);
Ac-(SEQ ID NO: 185) (herein referred to as BCY19305);
Ac-(SEQ ID NO: 186) (herein referred to as BCY19309);
Ac-(SEQ ID NO: 187)-[K(PYA)] (herein referred to as BCY19378);
Ac-(SEQ ID NO: 188) (herein referred to as BCY19533);
Ac-(SEQ ID NO: 189) (herein referred to as BCY19534);
Ac-(SEQ ID NO: 190) (herein referred to as BCY19535);
Ac-(SEQ ID NO: 191) (herein referred to as BCY19536);
Ac-(SEQ ID NO: 192) (herein referred to as BCY19537);
Ac-(SEQ ID NO: 193) (herein referred to as BCY19538);
Ac-(SEQ ID NO: 194) (herein referred to as BCY19539);
Ac-(SEQ ID NO: 195) (herein referred to as BCY19541);
Ac-(SEQ ID NO: 196) (herein referred to as BCY19542);
Ac-(SEQ ID NO: 197) (herein referred to as BCY19543);
Ac-(SEQ ID NO: 198) (herein referred to as BCY19544);
Ac-(SEQ ID NO: 199) (herein referred to as BCY19545);
Ac-(SEQ ID NO: 200) (herein referred to as BCY19546);
Ac-(SEQ ID NO: 201) (herein referred to as BCY19547);
Ac-(SEQ ID NO: 202) (herein referred to as BCY19548);
Ac-(SEQ ID NO: 203)-[K(PYA)] (herein referred to as BCY19599);
Ac-(SEQ ID NO: 204)-[K(PYA)] (herein referred to as BCY19600);
Ac-(SEQ ID NO: 204)-[K(PYA)]-triazolyl-PEG₁₀-amido-PIB (herein referred to as BCY20014);
Ac-(SEQ ID NO: 205)-[K(PYA)] (herein referred to as BCY19638);
Ac-(SEQ ID NO: 206)-[K(PYA)] (herein referred to as BCY19639);
Ac-(SEQ ID NO: 207)-[K(PYA)] (herein referred to as BCY19640);
Ac-(SEQ ID NO: 208)-[K(PYA)] (herein referred to as BCY19641);
Ac-(SEQ ID NO: 209)-[K(PYA)] (herein referred to as BCY19654);
Ac-(SEQ ID NO: 210)-[K(PYA)] (herein referred to as BCY19655);
Ac-(SEQ ID NO: 211)-[K(PYA)] (herein referred to as BCY19658);
Ac-(SEQ ID NO: 212)-[K(PYA)] (herein referred to as BCY19827);
Ac-(SEQ ID NO: 213)-[K(PYA)] (herein referred to as BCY19990); and
Ac-(SEQ ID NO: 214)-[K(PYA)] (herein referred to as BCY20268),
wherein PYA represents pentynoic acid and PIB represents 4 (4-iodophenyl) butyrate;
or wherein the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 37)-A-[Sar₆]-[KFl] (herein referred to as BCY15318);
Ac-(SEQ ID NO: 37)-[Sar₆]-[KFl] (herein referred to as BCY16323);
A-(SEQ ID NO: 38)-A-[Sar₆]-[KFl] (herein referred to as BCY15319); and
Ac-(SEQ ID NO: 88)-A-[Sar₆]-[KFl] (herein referred to as BCY16679).

16. The peptide ligand according to claim 1, wherein said loop sequences comprise three reactive groups separated by two loop sequences one of which consists of 8 amino acids and the other of which consists of 3 amino acids, such as:
wherein the bicyclic peptide ligand comprises an amino acid sequence which is selected from:
(SEQ ID NO: 45)
CᵢSNTWHWTDCᵢᵢLAECᵢᵢᵢ;
and
(SEQ ID NO: 47)
CᵢNLWNGDPWCᵢᵢLLRCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N- and/or C-terminal additions and comprises an amino acid sequence which is selected from:
A-(SEQ ID NO: 45)-A (herein referred to as BCY15351); and
A-(SEQ ID NO: 47)-A (herein referred to as BCY15353);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 45)-A-[Sar₆]-[KFl] (herein referred to as BCY15322); and
A-(SEQ ID NO: 47)-A-[Sar₆]-[KFl] (herein referred to as BCY15323).

17. The peptide ligand according to claim 1, wherein said loop sequences comprise three reactive groups separated by two loop sequences one of which consists of 8 amino acids and the other of which consists of 4 amino acids, such as:
wherein the bicyclic peptide ligand comprises an amino acid sequence which is selected from:
(SEQ ID NO: 42)
CᵢHQLMDIWDCᵢᵢLRPDCᵢᵢᵢ;
and
(SEQ ID NO: 43)
CᵢLTAREKIQCᵢᵢLQRRCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TCMT, the bicyclic peptide ligand additionally comprises N- and/or C-terminal additions and comprises an amino acid sequence which is selected from:
A-(SEQ ID NO: 42)-A (herein referred to as BCY15348); and
A-(SEQ ID NO: 43)-A (herein referred to as BCY15349);
or wherein the molecular scaffold is TCMT, the bicyclic peptide additionally comprises N- and/or C-terminal additions and a labelling moiety, such as fluorescein (Fl), and comprises an amino acid sequence which is selected from:
A-(SEQ ID NO: 42)-A-[Sar₆]-[KFl] (herein referred to as BCY15320); and
A-(SEQ ID NO: 43)-A-[Sar₆]-[KFl] (herein referred to as BCY15321).

18. The peptide ligand according to claim 1, which is selected from BCY15324, BCY16679, BCY15299, BCY15437, BCY15310, BCY16298 and BCY16287.

19. The peptide ligand according to claim 1, wherein the pharmaceutically acceptable salt is selected from the free acid or the sodium, potassium, calcium and ammonium salt.

20. A pharmaceutical composition which comprises the peptide ligand of claim 1, in combination with one or more pharmaceutically acceptable excipients.

21. The pharmaceutical composition according to claim 20, which additionally comprises one or more therapeutic agents.

22. A method for suppressing or treating a disease or disorder mediated by infection of SARS-COV-2 in a subject, or for providing prophylaxis to a subject at risk of infection of SARS-COV-2, such as COVID-19, comprising administering to the subject the peptide ligand according to claim 1.


SEQUENCE LISTING (214 sequences)

SEQ ID NO: 1 - protein, 12 aa, Artificial
  Synthetic Peptide
CHHACPILTGWC

SEQ ID NO: 2 - protein, 12 aa, Artificial
  Synthetic Peptide
CEDHDWVYCSTC

SEQ ID NO: 3 - protein, 15 aa, Artificial
  Synthetic Peptide
CVDANCKIKILQRMC

SEQ ID NO: 4 - protein, 15 aa, Artificial
  Synthetic Peptide
CTSSVCKIKELQRKC

SEQ ID NO: 5 - protein, 15 aa, Artificial
  Synthetic Peptide
CRSLLCEYLQRTDSC

SEQ ID NO: 6 - protein, 12 aa, Artificial
  Synthetic Peptide
CPHACPSLWGWC

SEQ ID NO: 7 - protein, 12 aa, Artificial
  Synthetic Peptide
CLHACPRLTHWC

SEQ ID NO: 8 - protein, 12 aa, Artificial
  Synthetic Peptide
CLHACQYLWGYC

SEQ ID NO: 9 - protein, 12 aa, Artificial
  Synthetic Peptide
CSHACPRLFGWC

SEQ ID NO: 10 - protein, 12 aa, Artificial
  Synthetic Peptide
CQHACPYLWDYC

SEQ ID NO: 11 - protein, 13 aa, Artificial
  Synthetic Peptide
CEYQGPHCYRLYC

SEQ ID NO: 12 - protein, 13 aa, Artificial
  Synthetic Peptide
CGRDSSWIYCSTC

SEQ ID NO: 13 - protein, 13 aa, Artificial
  Synthetic Peptide
CRGTPAWKACAIC

SEQ ID NO: 14 - protein, 15 aa, Artificial
  Synthetic Peptide
CLTKSCKIKMLQRVC

SEQ ID NO: 15 - protein, 15 aa, Artificial
  Synthetic Peptide
CMQPSCRVLQLQRVC

SEQ ID NO: 16 - protein, 15 aa, Artificial
  Synthetic Peptide
CALPSCRILHLQHRC

SEQ ID NO: 17 - protein, 15 aa, Artificial
  Synthetic Peptide
CHDAHCKILELQHRC

SEQ ID NO: 18 - protein, 15 aa, Artificial
  Synthetic Peptide
CTSSHCRVLEEQRLC

SEQ ID NO: 19 - protein, 15 aa, Artificial
  Synthetic Peptide
CPRDRCPTAWLYGLC

SEQ ID NO: 20 - protein, 15 aa, Artificial
  Synthetic Peptide
CAEAGCRVKQLQQIC

SEQ ID NO: 21 - protein, 15 aa, Artificial
  Synthetic Peptide
CTPSPCRVKELQRAC

SEQ ID NO: 22 - protein, 13 aa, Artificial
  Synthetic Peptide
CASPDNPVCRFYC

SEQ ID NO: 23 - protein, 12 aa, Artificial
  Synthetic Peptide
CAPWNYFRCDLC

SEQ ID NO: 24 - protein, 13 aa, Artificial
  Synthetic Peptide
CYNHANPVCRYYC

SEQ ID NO: 25 - protein, 12 aa, Artificial
  Synthetic Peptide
CLTPEDIWCMLC

SEQ ID NO: 26 - protein, 15 aa, Artificial
  Synthetic Peptide
CSTANCRILELQQLC

SEQ ID NO: 27 - protein, 13 aa, Artificial
  Synthetic Peptide
CDLFLHELCDMPC

SEQ ID NO: 28 - protein, 12 aa, Artificial
  Synthetic Peptide
CENPVDIWCVLC

SEQ ID NO: 29 - protein, 13 aa, Artificial
  Synthetic Peptide
CLTNDCHSDIRYC

SEQ ID NO: 30 - protein, 13 aa, Artificial
  Synthetic Peptide
CITNDCHTSLIFC

SEQ ID NO: 31 - protein, 13 aa, Artificial
  Synthetic Peptide
CNKQNWRYCYLTC

SEQ ID NO: 32 - protein, 15 aa, Artificial
  Synthetic Peptide
CTTSEKVKCLQRHPC

SEQ ID NO: 33 - protein, 15 aa, Artificial
  Synthetic Peptide
CQPDMRIKCLQRVAC

SEQ ID NO: 34 - protein, 15 aa, Artificial
  Synthetic Peptide
CSSNNRIKCLQRVTC

SEQ ID NO: 35 - protein, 15 aa, Artificial
  Synthetic Peptide
CKEKTTIGCLMAGIC

SEQ ID NO: 36 - protein, 13 aa, Artificial
  Synthetic Peptide
CPFPSGFGTCTFC

SEQ ID NO: 37 - protein, 13 aa, Artificial
  Synthetic Peptide
CPYVAGRGTCLLC

SEQ ID NO: 38 - protein, 13 aa, Artificial
  Synthetic Peptide
CPYPRGTGSCTFC

SEQ ID NO: 39 - protein, 13 aa, Artificial
  Synthetic Peptide
CLYPPGKGTCLLC

SEQ ID NO: 40 - protein, 13 aa, Artificial
  Synthetic Peptide
CPSPAGRGTCLLC

SEQ ID NO: 41 - protein, 13 aa, Artificial
  Synthetic Peptide
CPATIGRGPCTFC

SEQ ID NO: 42 - protein, 15 aa, Artificial
  Synthetic Peptide
CHQLMDIWDCLRPDC

SEQ ID NO: 43 - protein, 15 aa, Artificial
  Synthetic Peptide
CLTAREKIQCLQRRC

SEQ ID NO: 44 - protein, 15 aa, Artificial
  Synthetic Peptide
CVGRLCSTATDIRKC

SEQ ID NO: 45 - protein, 14 aa, Artificial
  Synthetic Peptide
CSNTWHWTDCLAEC

SEQ ID NO: 46 - protein, 12 aa, Artificial
  Synthetic Peptide
CVFTTVWDCLAC

SEQ ID NO: 47 - protein, 14 aa, Artificial
  Synthetic Peptide
CNLWNGDPWCLLRC

SEQ ID NO: 48 - protein, 15 aa, Artificial
  Synthetic Peptide
CRQSQCDWWAIRSFC

SEQ ID NO: 49 - protein, 15 aa, Artificial
  Synthetic Peptide
CTDATCSIKRLQRLC

SEQ ID NO: 50 - protein, 15 aa, Artificial
  Synthetic Peptide
CSPVSCPSGFKFGLC

SEQ ID NO: 51 - protein, 12 aa, Artificial
  Synthetic Peptide
CYDPIDVWCMMC

SEQ ID NO: 52 - protein, 12 aa, Artificial
  Synthetic Peptide
CASYDDFWCVLC

SEQ ID NO: 53 - protein, 12 aa, Artificial
  Synthetic Peptide
CDLTQHWTCILC

SEQ ID NO: 54 - protein, 12 aa, Artificial
  Synthetic Peptide
CSEISDVWCMLC

SEQ ID NO: 55 - protein, 12 aa, Artificial
  Synthetic Peptide
CPTPVDIWCMLC

SEQ ID NO: 56 - protein, 13 aa, Artificial
  Synthetic Peptide
CHPWSALFCNYPC

SEQ ID NO: 57 - protein, 13 aa, Artificial
  Synthetic Peptide
CYAPDNPVCRMYC

SEQ ID NO: 58 - protein, 12 aa, Artificial
  Synthetic Peptide
CPFACHKLYGWC

SEQ ID NO: 59 - protein, 12 aa, Artificial
  Synthetic Peptide
CMKACPYLYGWC

SEQ ID NO: 60 - protein, 12 aa, Artificial
  Synthetic Peptide
CRHACTHLYGHC

SEQ ID NO: 61 - protein, 12 aa, Artificial
  Synthetic Peptide
CPYACTRLYGWC

SEQ ID NO: 62 - protein, 12 aa, Artificial
  Synthetic Peptide
CSHACPRLTGWC

SEQ ID NO: 63 - protein, 12 aa, Artificial
  Synthetic Peptide
CLHSCPRLSGWC

SEQ ID NO: 64 - protein, 12 aa, Artificial
  Synthetic Peptide
CRHSCPILTGWC

SEQ ID NO: 65 - protein, 12 aa, Artificial
  Synthetic Peptide
CGHSCPVLWGWC

SEQ ID NO: 66 - protein, 12 aa, Artificial
  Synthetic Peptide
CPHSCPKLFGWC

SEQ ID NO: 67 - protein, 12 aa, Artificial
  Synthetic Peptide
CTHSCPYLFGWC

SEQ ID NO: 68 - protein, 15 aa, Artificial
  Synthetic Peptide
CDSPWCRIRSLQRQC

SEQ ID NO: 69 - protein, 15 aa, Artificial
  Synthetic Peptide
CSVGACRVKLLQRVC

SEQ ID NO: 70 - protein, 15 aa, Artificial
  Synthetic Peptide
CMFVPCAVREILGLC

SEQ ID NO: 71 - protein, 12 aa, Artificial
  Synthetic Peptide
CTLMDPWCLLKC

SEQ ID NO: 72 - protein, 12 aa, Artificial
  Synthetic Peptide
CKIHDWTCLLRC

SEQ ID NO: 73 - protein, 12 aa, Artificial
  Synthetic Peptide
CEQNGWIYCSTC

SEQ ID NO: 74 - protein, 12 aa, Artificial
  Synthetic Peptide
CTDRSWIFCSTC

SEQ ID NO: 75 - protein, 12 aa, Artificial
  Synthetic Peptide
CPNISWIYCSTC

SEQ ID NO: 76 - protein, 13 aa, Artificial
  Synthetic Peptide
CGILADPFCLISC

SEQ ID NO: 77 - protein, 13 aa, Artificial
  Synthetic Peptide
CPEANSWVYCSTC

SEQ ID NO: 78 - protein, 13 aa, Artificial
  Synthetic Peptide
CAPTSGWIYCSTC

SEQ ID NO: 79 - protein, 12 aa, Artificial
  Synthetic Peptide
CIPLDWTCMIAC

SEQ ID NO: 80 - protein, 13 aa, Artificial
  Synthetic Peptide
  Xaa (7)..(7): Xaa is Agb
CPYVAGXGTCLLC

SEQ ID NO: 81 - protein, 13 aa, Artificial
  Synthetic Peptide
  Xaa (7)..(7): Xaa is Arg(Me)
CPYVAGXGTCLLC

SEQ ID NO: 82 - protein, 13 aa, Artificial
  Synthetic Peptide
  Xaa (12)..(12): Xaa is Cba
CPYVAGRGTCLXC

SEQ ID NO: 83 - protein, 13 aa, Artificial
  Synthetic Peptide
  Xaa (11)..(11): Xaa is Cba
CPYVAGRGTCXLC

SEQ ID NO: 84 - protein, 13 aa, Artificial
  Synthetic Peptide
CPYVAGRATCLLC

SEQ ID NO: 85 - protein, 13 aa, Artificial
  Synthetic Peptide
  Xaa (7)..(7): Xaa is HArg
CPYVAGXGTCLLC

SEQ ID NO: 86 - protein, 13 aa, Artificial
  Synthetic Peptide
  Xaa (12)..(12): Xaa is tBuAla
CPYVAGRGTCLXC

SEQ ID NO: 87 - protein, 13 aa, Artificial
  Synthetic Peptide
  Xaa (11)..(11): Xaa is tBuAla
CPYVAGRGTCXLC

SEQ ID NO: 88 - protein, 13 aa, Artificial
  Synthetic Peptide
  Xaa (7)..(7): Xaa is Agb
  Xaa (12)..(12): Xaa is tBuAla
CPYVAGXATCLXC

SEQ ID NO: 89 - protein, 13 aa, Artificial
  Synthetic Peptide
  Xaa (10)..(10): Xaa is Agb
CYNHANPVCXYYC

SEQ ID NO: 90 - protein, 13 aa, Artificial
  Synthetic Peptide
  Xaa (10)..(10): Xaa is Agb
CASPDNPVCXFYC

SEQ ID NO: 91 - protein, 13 aa, Artificial
  Synthetic Peptide
  Xaa (10)..(10): Xaa is Arg(Me)
CASPDNPVCXFYC

SEQ ID NO: 92 - protein, 13 aa, Artificial
  Synthetic Peptide
  Xaa (10)..(10): Xaa is HArg
CASPDNPVCXFYC

SEQ ID NO: 93 - protein, 13 aa, Artificial
  Synthetic Peptide
CANPDNPVCRFYC

SEQ ID NO: 94 - protein, 13 aa, Artificial
  Synthetic Peptide
CRNPDNPVCRFYC

SEQ ID NO: 95 - protein, 13 aa, Artificial
  Synthetic Peptide
CHNPSNPVCRFYC

SEQ ID NO: 96 - protein, 13 aa, Artificial
  Synthetic Peptide
CVNKHNPVCRFYC

SEQ ID NO: 97 - protein, 13 aa, Artificial
  Synthetic Peptide
CVNAENPVCRFYC

SEQ ID NO: 98 - protein, 13 aa, Artificial
  Synthetic Peptide
CQNPGNPVCRFYC

SEQ ID NO: 99 - protein, 13 aa, Artificial
  Synthetic Peptide
CMNPDNPVCRFYC

SEQ ID NO: 100 - protein, 13 aa, Artificial
  Synthetic Peptide
CYNQENPVCRFYC

SEQ ID NO: 101 - protein, 13 aa, Artificial
  Synthetic Peptide
CNNPANPVCRFYC

SEQ ID NO: 102 - protein, 13 aa, Artificial
  Synthetic Peptide
CFNIDNPVCRFYC

SEQ ID NO: 103 - protein, 13 aa, Artificial
  Synthetic Peptide
CSNPENPVCRFYC

SEQ ID NO: 104 - protein, 13 aa, Artificial
  Synthetic Peptide
CMNEDNPVCRFYC

SEQ ID NO: 105 - protein, 13 aa, Artificial
  Synthetic Peptide
CMNEANPVCRFYC

SEQ ID NO: 106 - protein, 13 aa, Artificial
  Synthetic Peptide
CHNLDNPVCRFYC

SEQ ID NO: 107 - protein, 13 aa, Artificial
  Synthetic Peptide
CANHDNPVCRFYC

SEQ ID NO: 108 - protein, 13 aa, Artificial
  Synthetic Peptide
CKNYDNPVCRFYC

SEQ ID NO: 109 - protein, 13 aa, Artificial
  Synthetic Peptide
CENMDNPVCRFYC

SEQ ID NO: 110 - protein, 13 aa, Artificial
  Synthetic Peptide
CMNTDNPVCRFYC

SEQ ID NO: 111 - protein, 13 aa, Artificial
  Synthetic Peptide
CLNVDNPVCRFYC

SEQ ID NO: 112 - protein, 13 aa, Artificial
  Synthetic Peptide
CLNPDNPVCRFYC

SEQ ID NO: 113 - protein, 13 aa, Artificial
  Synthetic Peptide
  Xaa (10)..(10): Xaa is HArg
CYNHANPVCXYYC

SEQ ID NO: 114 - protein, 13 aa, Artificial
  Synthetic Peptide
  Xaa (10)..(10): Xaa is Arg(Me)
CYNHANPVCXYYC

SEQ ID NO: 115 - protein, 12 aa, Artificial
  Synthetic Peptide
CFDDWTCYIQMC

SEQ ID NO: 116 - protein, 13 aa, Artificial
  Synthetic Peptide
CMNPFFYDCERTC

SEQ ID NO: 117 - protein, 12 aa, Artificial
  Synthetic Peptide
CDVCGLNAFNRC

SEQ ID NO: 118 - protein, 12 aa, Artificial
  Synthetic Peptide
CDWTCYLTMMPC

SEQ ID NO: 119 - protein, 12 aa, Artificial
  Synthetic Peptide
CDWTCYLRPLPC

SEQ ID NO: 120 - protein, 12 aa, Artificial
  Synthetic Peptide
CDWTCYMSMKPC

SEQ ID NO: 121 - protein, 12 aa, Artificial
  Synthetic Peptide
CDWTCYFRPLPC

SEQ ID NO: 122 - protein, 13 aa, Artificial
  Synthetic Peptide
CDWTCYISPMFDC

SEQ ID NO: 123 - protein, 13 aa, Artificial
  Synthetic Peptide
CDWTCYLNIYHEC

SEQ ID NO: 124 - protein, 13 aa, Artificial
  Synthetic Peptide
CDWTCYMDYLSNC

SEQ ID NO: 125 - protein, 13 aa, Artificial
  Synthetic Peptide
CDWTCYLRIHEAC

SEQ ID NO: 126 - protein, 13 aa, Artificial
  Synthetic Peptide
CDWTCYMRINDAC

SEQ ID NO: 127 - protein, 13 aa, Artificial
  Synthetic Peptide
CDWTCYINIYNTC

SEQ ID NO: 128 - protein, 15 aa, Artificial
  Synthetic Peptide
CSDLMCWYLQRTDSC

SEQ ID NO: 129 - protein, 15 aa, Artificial
  Synthetic Peptide
CNSYMCWYLQRTDSC

SEQ ID NO: 130 - protein, 15 aa, Artificial
  Synthetic Peptide
CTSYLCWYLQRTDSC

SEQ ID NO: 131 - protein, 15 aa, Artificial
  Synthetic Peptide
CRSLMCWYLNQTDSC

SEQ ID NO: 132 - protein, 13 aa, Artificial
  Synthetic Peptide
CEYNGPYCYRLYC

SEQ ID NO: 133 - protein, 13 aa, Artificial
  Synthetic Peptide
CEYVGPMCYRLYC

SEQ ID NO: 134 - protein, 12 aa, Artificial
  Synthetic Peptide
CLDETWIYCSTC

SEQ ID NO: 135 - protein, 12 aa, Artificial
  Synthetic Peptide
CPDETWVYCSTC

SEQ ID NO: 136 - protein, 12 aa, Artificial
  Synthetic Peptide
CESNDWVYCSTC

SEQ ID NO: 137 - protein, 12 aa, Artificial
  Synthetic Peptide
CEDNDWVYCSTC

SEQ ID NO: 138 - protein, 12 aa, Artificial
  Synthetic Peptide
CPDVSWIYCSTC

SEQ ID NO: 139 - protein, 13 aa, Artificial
  Synthetic Peptide
  Xaa (2)..(2): Xaa is Aib
CXSPDNPVCRFYC

SEQ ID NO: 140 - protein, 13 aa, Artificial
  Synthetic Peptide
  Xaa (4)..(4): Xaa is HyP
CASXDNPVCRFYC

SEQ ID NO: 141 - protein, 13 aa, Artificial
  Synthetic Peptide
  Xaa (4)..(4): Xaa is Aib
CASXDNPVCRFYC

SEQ ID NO: 142 - protein, 13 aa, Artificial
  Synthetic Peptide
  Xaa (4)..(4): Xaa is Pip
CASXDNPVCRFYC

SEQ ID NO: 143 - protein, 13 aa, Artificial
  Synthetic Peptide
  Xaa (7)..(7): Xaa is Pip
CASPDNXVCRFYC

SEQ ID NO: 144 - protein, 13 aa, Artificial
  Synthetic Peptide
  Xaa (7)..(7): Xaa is 44DFP
CASPDNXVCRFYC

SEQ ID NO: 145 - protein, 13 aa, Artificial
  Synthetic Peptide
  Xaa (7)..(7): Xaa is 4FlPro
CASPDNXVCRFYC

SEQ ID NO: 146 - protein, 13 aa, Artificial
  Synthetic Peptide
  Xaa (11)..(11): Xaa is 1Nal
CASPDNPVCRXYC

SEQ ID NO: 147 - protein, 13 aa, Artificial
  Synthetic Peptide
  Xaa (11)..(11): Xaa is 2Nal
CASPDNPVCRXYC

SEQ ID NO: 148 - protein, 13 aa, Artificial
  Synthetic Peptide
  Xaa (11)..(11): Xaa is 2MePhe
CASPDNPVCRXYC

SEQ ID NO: 149 - protein, 13 aa, Artificial
  Synthetic Peptide
  Xaa (11)..(11): Xaa is 3MePhe
CASPDNPVCRXYC

SEQ ID NO: 150 - protein, 12 aa, Artificial
  Synthetic Peptide
  Xaa (11)..(11): Xaa is 4MePhe
CASPDNPVCRXC

SEQ ID NO: 151 - protein, 13 aa, Artificial
  Synthetic Peptide
  Xaa (11)..(11): Xaa is 2ClPhe
CASPDNPVCRXYC

SEQ ID NO: 152 - protein, 13 aa, Artificial
  Synthetic Peptide
  Xaa (11)..(11): Xaa is 3ClPhe
CASPDNPVCRXYC

SEQ ID NO: 153 - protein, 13 aa, Artificial
  Synthetic Peptide
  Xaa (11)..(11): Xaa is 4ClPhe
CASPDNPVCRXYC

SEQ ID NO: 154 - protein, 13 aa, Artificial
  Synthetic Peptide
  Xaa (11)..(11): Xaa is 2FPhe
CASPDNPVCRXYC

SEQ ID NO: 155 - protein, 13 aa, Artificial
  Synthetic Peptide
  Xaa (11)..(11): Xaa is 3FPhe
CASPDNPVCRXYC

SEQ ID NO: 156 - protein, 13 aa, Artificial
  Synthetic Peptide
  Xaa (11)..(11): Xaa is 4FPhe
CASPDNPVCRXYC

SEQ ID NO: 157 - protein, 13 aa, Artificial
  Synthetic Peptide
  Xaa (11)..(11): Xaa is 26DiMeTyr
CASPDNPVCRXYC

SEQ ID NO: 158 - protein, 13 aa, Artificial
  Synthetic Peptide
  Xaa (2)..(2): Xaa is Aib
  Xaa (7)..(7): Xaa is 44DFP
  Xaa (11)..(11): Xaa is 4FPhe
CXSPDNXVCRXYC

SEQ ID NO: 159 - protein, 13 aa, Artificial
  Synthetic Peptide
  Xaa (2)..(2): Xaa is Aib
  Xaa (7)..(7): Xaa is 44DFP
  Xaa (10)..(10): Xaa is Arg(Me)
  Xaa (11)..(11): Xaa is 4FPhe
CXSPDNXVCXXYC

SEQ ID NO: 160 - protein, 13 aa, Artificial
  Synthetic Peptide
  Xaa (2)..(2): Xaa is Aib
  Xaa (11)..(11): Xaa is 4FPhe
CXSPDNPVCRXYC

SEQ ID NO: 161 - protein, 13 aa, Artificial
  Synthetic Peptide
  Xaa (2)..(2): Xaa is Aib
  Xaa (10)..(10): Xaa is Arg(Me)
  Xaa (11)..(11): Xaa is 4FPhe
CXSPDNPVCXXYC

SEQ ID NO: 162 - protein, 13 aa, Artificial
  Synthetic Peptide
  Xaa (2)..(2): Xaa is Aib
  Xaa (7)..(7): Xaa is 44DFP
  Xaa (11)..(11): Xaa is 4FPhe
CXNPDNXVCRXYC

SEQ ID NO: 163 - protein, 13 aa, Artificial
  Synthetic Peptide
  Xaa (2)..(2): Xaa is Aib
  Xaa (7)..(7): Xaa is 44DFP
  Xaa (10)..(10): Xaa is Arg(Me)
  Xaa (11)..(11): Xaa is 4FPhe
CXNPDNXVCXXYC

SEQ ID NO: 164 - protein, 13 aa, Artificial
  Synthetic Peptide
  Xaa (2)..(2): Xaa is Aib
  Xaa (11)..(11): Xaa is 4FPhe
CXNPDNPVCRXYC

SEQ ID NO: 165 - protein, 13 aa, Artificial
  Synthetic Peptide
  Xaa (2)..(2): Xaa is Aib
  Xaa (10)..(10): Xaa is Arg(Me)
  Xaa (11)..(11): Xaa is 4FPhe
CXNPDNPVCXXYC

SEQ ID NO: 166 - protein, 15 aa, Artificial
  Synthetic Peptide
CNRPTSVYCLQRGIC

SEQ ID NO: 167 - protein, 13 aa, Artificial
  Synthetic Peptide
  Xaa (7)..(7): Xaa is HArg
  Xaa (12)..(12): Xaa is tBuAla
CPYVAGXATCLXC

SEQ ID NO: 168 - protein, 13 aa, Artificial
  Synthetic Peptide
  Xaa (7)..(7): Xaa is HArg
  Xaa (12)..(12): Xaa is tBuAla
CPYVPGXATCLXC

SEQ ID NO: 169 - protein, 13 aa, Artificial
  Synthetic Peptide
  Xaa (2)..(2): Xaa is K(PYA)
  Xaa (7)..(7): Xaa is HArg
  Xaa (12)..(12): Xaa is tBuAla
CXYVAGXATCLXC

SEQ ID NO: 170 - protein, 13 aa, Artificial
  Synthetic Peptide
  Xaa (2)..(2): Xaa is HyP
  Xaa (7)..(7): Xaa is HArg
  Xaa (12)..(12): Xaa is tBuAla
CXYVAGXATCLXC

SEQ ID NO: 171 - protein, 13 aa, Artificial
  Synthetic Peptide
  Xaa (12)..(12): Xaa is tBuAla
CPYVAGTATCLXC

SEQ ID NO: 172 - protein, 13 aa, Artificial
  Synthetic Peptide
  Xaa (7)..(7): Xaa is Agb
CPYVAGXATCLLC

SEQ ID NO: 173 - protein, 13 aa, Artificial
  Synthetic Peptide
  Xaa (7)..(7): Xaa is Agb
  Xaa (12)..(12): Xaa is tBuAla
CPYVAGXGTCLXC

SEQ ID NO: 174 - protein, 13 aa, Artificial
  Synthetic Peptide
  Xaa (5)..(5): Xaa is HyP
  Xaa (7)..(7): Xaa is HArg
  Xaa (12)..(12): Xaa is tBuAla
CPYVXGXATCLXC

SEQ ID NO: 175 - protein, 13 aa, Artificial
  Synthetic Peptide
  Xaa (4)..(4): Xaa is K(PYA)
  Xaa (7)..(7): Xaa is HArg
  Xaa (12)..(12): Xaa is tBuAla
CPYXAGXATCLXC

SEQ ID NO: 176 - protein, 13 aa, Artificial
  Synthetic Peptide
  Xaa (2)..(2): Xaa is Oic
  Xaa (7)..(7): Xaa is HArg
  Xaa (12)..(12): Xaa is tBuAla
CXYVAGXATCLXC

SEQ ID NO: 177 - protein, 13 aa, Artificial
  Synthetic Peptide
  Xaa (4)..(4): Xaa is B-Melle
  Xaa (7)..(7): Xaa is HArg
  Xaa (12)..(12): Xaa is tBuAla
CPYXAGXATCLXC

SEQ ID NO: 178 - protein, 13 aa, Artificial
  Synthetic Peptide
  Xaa (4)..(4): Xaa is tBuGly
  Xaa (7)..(7): Xaa is HArg
  Xaa (12)..(12): Xaa is tBuAla
CPYXAGXATCLXC

SEQ ID NO: 179 - protein, 13 aa, Artificial
  Synthetic Peptide
  Xaa (7)..(7): Xaa is HArg
  Xaa (12)..(12): Xaa is tBuAla
CPYPAGXATCLXC

SEQ ID NO: 180 - protein, 13 aa, Artificial
  Synthetic Peptide
  Xaa (3)..(3): Xaa is 44BPA
  Xaa (7)..(7): Xaa is HArg
  Xaa (12)..(12): Xaa is tBuAla
CPXVAGXATCLXC

SEQ ID NO: 181 - protein, 13 aa, Artificial
  Synthetic Peptide
  Xaa (3)..(3): Xaa is 2FPhe
  Xaa (7)..(7): Xaa is HArg
  Xaa (12)..(12): Xaa is tBuAla
CPXVAGXATCLXC

SEQ ID NO: 182 - protein, 13 aa, Artificial
  Synthetic Peptide
  Xaa (4)..(4): Xaa is Cba
  Xaa (7)..(7): Xaa is HArg
  Xaa (12)..(12): Xaa is tBuAla
CPYXAGXATCLXC

SEQ ID NO: 183 - protein, 13 aa, Artificial
  Synthetic Peptide
  Xaa (3)..(3): Xaa is 3FPhe
  Xaa (7)..(7): Xaa is HArg
  Xaa (12)..(12): Xaa is tBuAla
CPXVAGXATCLXC

SEQ ID NO: 184 - protein, 13 aa, Artificial
  Synthetic Peptide
  Xaa (2)..(2): Xaa is 55DMP
  Xaa (7)..(7): Xaa is HArg
  Xaa (12)..(12): Xaa is tBuAla
CXYVAGXATCLXC

SEQ ID NO: 185 - protein, 13 aa, Artificial
  Synthetic Peptide
  Xaa (12)..(12): Xaa is tBuAla
CPYVAGQATCLXC

SEQ ID NO: 186 - protein, 13 aa, Artificial
  Synthetic Peptide
  Xaa (7)..(7): Xaa is HArg
  Xaa (12)..(12): Xaa is tBuAla
CPYVAGXSTCLXC

SEQ ID NO: 187 - protein, 13 aa, Artificial
  Synthetic Peptide
  Xaa (3)..(3): Xaa is 4tBuPhe
  Xaa (7)..(7): Xaa is HArg
  Xaa (12)..(12): Xaa is tBuAla
CPXVAGXATCLXC

SEQ ID NO: 188 - protein, 13 aa, Artificial
  Synthetic Peptide
CPYREGTGTCLLC

SEQ ID NO: 189 - protein, 13 aa, Artificial
  Synthetic Peptide
CPYAPGNGTCLLC

SEQ ID NO: 190 - protein, 13 aa, Artificial
  Synthetic Peptide
CPHPPGRGTCLLC

SEQ ID NO: 191 - protein, 13 aa, Artificial
  Synthetic Peptide
CPYNAGTGTCLLC

SEQ ID NO: 192 - protein, 13 aa, Artificial
  Synthetic Peptide
CPYSPGQGTCLLC

SEQ ID NO: 193 - protein, 13 aa, Artificial
  Synthetic Peptide
CPYQPGSGTCLLC

SEQ ID NO: 194 - protein, 13 aa, Artificial
  Synthetic Peptide
CPFPPGMGTCLLC

SEQ ID NO: 195 - protein, 13 aa, Artificial
  Synthetic Peptide
CPHQPGFGTCLLC

SEQ ID NO: 196 - protein, 13 aa, Artificial
  Synthetic Peptide
CPYSPGSGTCLLC

SEQ ID NO: 197 - protein, 13 aa, Artificial
  Synthetic Peptide
CPYLAGTGTCLLC

SEQ ID NO: 198 - protein, 13 aa, Artificial
  Synthetic Peptide
CPWEAGKGTCLLC

SEQ ID NO: 199 - protein, 13 aa, Artificial
  Synthetic Peptide
CPYAPGMGTCLLC

SEQ ID NO: 200 - protein, 13 aa, Artificial
  Synthetic Peptide
CPHMPGSGTCLLC

SEQ ID NO: 201 - protein, 13 aa, Artificial
  Synthetic Peptide
CPYNKGEGTCLLC

SEQ ID NO: 202 - protein, 13 aa, Artificial
  Synthetic Peptide
CPFKPGVGTCLLC

SEQ ID NO: 203 - protein, 13 aa, Artificial
  Synthetic Peptide
  Xaa (3)..(3): Xaa is 4tBuPhe
  Xaa (7)..(7): Xaa is Orn
  Xaa (12)..(12): Xaa is tBuAla
CPXVAGXATCLXC

SEQ ID NO: 204 - protein, 13 aa, Artificial
  Synthetic Peptide
  Xaa (2)..(2): Xaa is Oic
  Xaa (3)..(3): Xaa is 4tBuPhe
  Xaa (7)..(7): Xaa is HArg
  Xaa (12)..(12): Xaa is tBuAla
CXXVAGXATCLXC

SEQ ID NO: 205 - protein, 13 aa, Artificial
  Synthetic Peptide
  Xaa (3)..(3): Xaa is 4tBuPhe
  Xaa (7)..(7): Xaa is Dab
  Xaa (12)..(12): Xaa is tBuAla
CPXVAGXATCLXC

SEQ ID NO: 206 - protein, 13 aa, Artificial
  Synthetic Peptide
  Xaa (3)..(3): Xaa is 4tBuPhe
  Xaa (7)..(7): Xaa is Dap
  Xaa (12)..(12): Xaa is tBuAla
CPXVAGXATCLXC

SEQ ID NO: 207 - protein, 13 aa, Artificial
  Synthetic Peptide
  Xaa (3)..(3): Xaa is 4CF3Phe
  Xaa (7)..(7): Xaa is HArg
  Xaa (12)..(12): Xaa is tBuAla
CPXVAGXATCLXC

SEQ ID NO: 208 - protein, 13 aa, Artificial
  Synthetic Peptide
  Xaa (3)..(3): Xaa is DMAPhe
  Xaa (7)..(7): Xaa is HArg
  Xaa (12)..(12): Xaa is tBuAla
CPXVAGXATCLXC

SEQ ID NO: 209 - protein, 13 aa, Artificial
  Synthetic Peptide
  Xaa (3)..(3): Xaa is 4tBuPhe
  Xaa (7)..(7): Xaa is HArg
  Xaa (8)..(8): Xaa is dDab
  Xaa (12)..(12): Xaa is tBuAla
CPXVAGXXTCLXC

SEQ ID NO: 210 - protein, 13 aa, Artificial
  Synthetic Peptide
  Xaa (3)..(3): Xaa is 4tBuPhe
  Xaa (7)..(7): Xaa is HSer
  Xaa (12)..(12): Xaa is tBuAla
CPXVAGXATCLXC

SEQ ID NO: 211 - protein, 13 aa, Artificial
  Synthetic Peptide
  Xaa (3)..(3): Xaa is 4tBuPhe
  Xaa (7)..(7): Xaa is Cit
  Xaa (12)..(12): Xaa is tBuAla
CPXVAGXATCLXC

SEQ ID NO: 212 - protein, 13 aa, Artificial
  Synthetic Peptide
  Xaa (2)..(2): Xaa is Oic
  Xaa (3)..(3): Xaa is 4tBuPhe
  Xaa (7)..(7): Xaa is Orn
  Xaa (12)..(12): Xaa is tBuAla
CXXVAGXATCLXC

SEQ ID NO: 213 - protein, 13 aa, Artificial
  Synthetic Peptide
  Xaa (3)..(3): Xaa is 4tBuPhe
  Xaa (7)..(7): Xaa is HArg
  Xaa (8)..(8): Xaa is dDap
  Xaa (12)..(12): Xaa is tBuAla
CPXVAGXXTCLXC

SEQ ID NO: 214 - protein, 13 aa, Artificial
  Synthetic Peptide
  Xaa (2)..(2): Xaa is Oic
  Xaa (3)..(3): Xaa is 4tBuPhe
  Xaa (7)..(7): Xaa is Cit
  Xaa (12)..(12): Xaa is tBuAla
CXXVAGXATCLXC